(19) United States
(12) Patent Application Publication
(10) Pub. No.: US 2024/0028782 A1
(43) Pub. Date: Jan. 25, 2024

(54) DENTAL RESTORATION AUTOMATION

(71) Applicant: James R. Glidewell Dental Ceramics, Inc., Newport Beach, CA (US)

(72) Inventors: Michael J. Selberis, Ladera Ranch, CA (US); Sergei Azernikov, Irvine, CA (US); David Christopher Leeson, Laguna Beach, CA (US); Maksim Malkov, Newport Beach, CA (US)

(73) Assignee: James R. Glidewell Dental Ceramics, Inc., Newport Beach, CA (US)

(21) Appl. No.: 18/353,947

(22) Filed: Jul. 18, 2023

Related U.S. Application Data
(60) Provisional application No. 63/369,151, filed on Jul. 22, 2022.
(60) Provisional application No. 63/380,374, filed on Oct. 20, 2022.

(51) Int. Cl. G06F 30/10; A61C 13/34

(52) U.S. Cl. CPC G06F 30/10; A61C 13/34

(57) ABSTRACT

A computer-implemented method and system of virtual dental restoration design automation includes: receiving a 3D virtual dental model of at least a portion of a patient's dentition, the 3D virtual dental model comprising at least one virtual preparation tooth, the virtual preparation tooth comprising a digital representation of a physical preparation tooth prepared by a dentist;

performing an automated virtual restoration design using the 3D virtual dental model; displaying virtually to the dentist one or more physical preparation tooth issues detected while performing the automated virtual restoration design; and displaying virtually to the dentist a generated virtual restoration for one or more adjustments where no physical preparation tooth issues are detected while performing the automated virtual restoration design.

The present application claims priority to and the benefit of co-pending U.S. Provisional Patent Application Ser. No. 63/369,151, entitled Integrated Dental Restoration Design Process and System, filed on Jul. 22, 2022, and to co-pending U.S. Provisional Patent Application Ser. 63/380,374, entitled Dental Restoration Automation, filed on Oct. 20, 2022, both of which are hereby incorporated by reference in their entirety.

### BACKGROUND

Traditional dental practice typically involves a dentist receiving a patient in their dental office and performing an examination of a patient's dentition. Examination/treatment in a dental office can be referred to as “chairside” since the patient is in the dental chair. During a typical chairside visit, the patient may require a dental restoration for one or more teeth. The dentist typically anesthetizes the patient or a portion of the patient's dentition to physically prepare the one or more teeth to receive a dental restoration. During this preparation chairside visit, the dentist can modify the one or more existing teeth in a way to allow a restoration to properly fit onto a particular prepared tooth or teeth. Examples of restorations can include—without limit—crowns, bridges, inlays, etc.

In some instances, once the physical tooth or teeth are prepared, the dentist may directly scan the patient's dentition that includes the physical preparation tooth/teeth to generate a 3D virtual model of the patient's dentition that includes a virtual preparation tooth representing the physically prepared tooth. In some cases, this scanning can be performed using an optical scanner such as an intraoral scanner, for example. This can generate a 3D virtual model of at least a portion of the patient's dentition including the preparation tooth/teeth.

In some cases, the dentist can have the patient create an impression by having the patient bite down into impression material arranged in an impression tray to generate a physical impression. This physical impression can be mailed to a dental laboratory where a plaster mold of the patient's dentition is made from the physical impression.

In some cases, a 3D physical plaster model of the patient's dentition can be fabricated from the physical impression and then scanned optically at the dental laboratory. Alternatively, the physical impression itself can be scanned using an optical scanner such as an intraoral scanner or a computerized tomographic (“CT”) scanner to generate a 3D virtual model of the patient's dentition including the preparation tooth/teeth at the dental laboratory.

Recently, CAD/CAM dentistry (Computer-Aided Design and Computer-Aided Manufacturing in dentistry) has provided a broad range of dental restorations, including crowns, veneers, inlays and onlays, fixed bridges, dental implant restorations, and orthodontic appliances. In a typical CAD/CAM based dental procedure, a treating dentist can prepare the tooth being restored either as a crown, inlay, onlay or veneer. The prepared tooth and its surroundings are then scanned by a three dimensional (3D) imaging camera and uploaded to a computer for design. Alternatively, a dentist can obtain an impression of the tooth to be restored and the impression may be scanned directly, or formed into a model to be scanned, and uploaded to a computer for design.

Current dental CAD can often be tedious, time-consuming, and can lead to inconsistencies in design and errors. In some cases, issues can arise that can prevent or hinder design of a restoration for the preparation tooth due to issues with the physical preparation tooth. These issues can include but are not limited to, for example, one or more undercut regions on the physical preparation tooth, margin line issues, one or more clearance issue regions, and/or restoration insertion issues. These issues can reoccur over time if not addressed. Detecting and addressing these issues with the dentist can be desirable, as can minimizing errors.

### SUMMARY

A computer-implemented method of virtual dental restoration design automation can include: receiving a 3D virtual dental model of at least a portion of a patient's dentition, the 3D virtual dental model comprising at least one virtual preparation tooth, the virtual preparation tooth comprising a digital representation of a physical preparation tooth prepared by a dentist; performing an automated virtual restoration design using the 3D virtual dental model; displaying virtually to the dentist one or more physical preparation tooth issues detected while performing the automated virtual restoration design; and displaying virtually to the dentist a generated virtual restoration for one or more adjustments where no physical preparation tooth issues are detected while performing the automated virtual restoration design.

A non-transitory computer readable medium storing executable computer program instructions to provide virtual dental restoration design automation, the computer program instructions can include instructions for: receiving a 3D virtual dental model of at least a portion of a patient's dentition, the 3D virtual dental model comprising at least one virtual preparation tooth, the virtual preparation tooth comprising a digital representation of a physical preparation tooth prepared by a dentist; performing an automated virtual restoration design using the 3D virtual dental model; displaying virtually to the dentist one or more physical preparation tooth issues detected while performing the automated virtual restoration design; and displaying virtually to the dentist a generated virtual restoration for one or more adjustments where no physical preparation tooth issues are detected while performing the automated virtual restoration design.

A system for virtual dental restoration design automation can include: a processor; and a non-transitory computer-readable storage medium comprising instructions executable by the processor to perform steps comprising: receiving a 3D virtual dental model of at least a portion of a patient's dentition, the 3D virtual dental model comprising at least one virtual preparation tooth, the virtual preparation tooth comprising a digital representation of a physical preparation tooth prepared by a dentist; performing an automated virtual restoration design using the 3D virtual dental model; displaying virtually to the dentist one or more physical preparation tooth issues detected while performing the automated virtual restoration design; and displaying virtually to the dentist a generated virtual restoration for one or more adjustments where no physical preparation tooth issues are detected while performing the automated virtual restoration design.

### BRIEF DESCRIPTION OF THE DRAWINGS

FIG. 1 shows a diagram a cloud computing environment of a dental restoration automation system in some embodiments.

FIG. 2 shows a diagram of dental restoration automation in some embodiments.

FIG. 3 shows a diagram of dental restoration automation in some embodiments.

FIG. 4 shows a diagram of one or more steps that can be used in automated design of a virtual dental restoration in some embodiments.

FIG. 5(a)-5(c) show decimation performed on a 3D virtual model mesh in some embodiments

FIG. 6 shows a diagram of a convolutional neural network in some embodiments for example.

FIG. 7 shows a top perspective view of an example of a 2D depth map of a digital model in some embodiments for example.

FIG. 8 shows a top perspective view of a 3D virtual model with an occlusion direction, a virtual preparation die region, and a buccal direction.

FIG. 9(a) and FIG. 9(b) show diagrams of a hierarchical neural network in some embodiments for example.

FIG. 10 shows a diagram of deep neural network in some embodiments for example.

FIG. 11 shows a diagram of a computer-implemented method of automatic margin line proposal in some embodiments for example.

FIG. 12 shows a perspective view of an example of a 3D digital model showing a proposed margin line from a base margin line in some embodiments, for example.

FIG. 13(a) and FIG. 13(b) show a perspective view of a 3D digital model with a preparation tooth and a proposed margin line in some embodiments for example.

FIG. 14 shows a 3D virtual model of a virtual preparation tooth with a margin line in some embodiments.

FIG. 15 is a flow chart of a process for generating a 3D dental prosthesis model using a deep neural network in accordance with some embodiments of the present disclosure.

FIG. 16 is a graphic representation of input and output to a deep neural network in accordance with some embodiments of the present disclosure.

FIG. 17(a) and FIG. 17(b) are flow diagrams of methods for training a deep neural network to generate a 3D dental prosthesis in accordance with some embodiments of the present disclosure.

FIG. 18 shows a 3D virtual model of a virtual crown lifted above a virtual preparation tooth along an insertion direction in some embodiments.

FIG. 19 shows a diagram of a computer-implemented method of automatic margin line proposal in some embodiments in some embodiments.

FIG. 20 shows a 3D virtual model of a virtual preparation tooth with an open margin line in some embodiments.

FIG. 21 shows a perspective view of a 3D virtual model with a virtual preparation tooth and a marked open margin in some embodiments.

FIG. 22 shows a perspective view of a 3D virtual model with a virtual preparation tooth and a marked clearance issue in some embodiments.

FIG. 23(a) through FIG. 23(g) show one or more adjustments made to a virtual restoration in a 3D virtual dental model.

FIG. 24 is a flowchart in some embodiments.

FIG. 25 is a diagram illustrating a computing system in some embodiments.

### DETAILED DESCRIPTION

For purposes of this description, certain aspects, advantages, and novel features of the embodiments of this disclosure are described herein. The disclosed methods, apparatus, and systems should not be construed as being limiting in any way. Instead, the present disclosure is directed toward all novel and nonobvious features and aspects of the various disclosed embodiments, alone and in various combinations and sub-combinations with one another. The methods, apparatus, and systems are not limited to any specific aspect or feature or combination thereof, nor do the disclosed embodiments require that any one or more specific advantages be present or problems be solved.

Although the operations of some of the disclosed embodiments are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the terms “coupled” and “associated” generally mean electrically, electromagnetically, and/or physically (e.g., mechanically or chemically) coupled or linked and does not exclude the presence of intermediate elements between the coupled or associated items absent specific contrary language.

In some examples, values, procedures, or apparatus may be referred to as “lowest,” “best,” “minimum,” or the like. It will be appreciated that such descriptions are intended to indicate that a selection among many alternatives can be made, and such selections need not be better, smaller, or otherwise preferable to other selections.

In the following description, certain terms may be used such as “up,” “down,” “upper,” “lower,” “horizontal,” “vertical,” “left,” “right,” and the like. These terms are used, where applicable, to provide some clarity of description when dealing with relative relationships. But, these terms are not intended to imply absolute relationships, positions, and/or orientations. For example, with respect to an object, an “upper” surface can become a “lower” surface simply by turning the object over. Nevertheless, it is still the same object.

All reference to any other U.S. applications and patents and any other publications are hereby incorporated by reference in their entirety.

As used herein, the term “dental restoration” can refer to any dental restorative (restoration) including, without limitation, crowns, bridges, dentures, partial dentures, implants, onlays, inlays, or veneers.

Some embodiments disclose a computer-implemented method of virtual dental restoration design automation. Some embodiments in the present disclosure can include a workflow that can perform one or more steps in an integrated virtual restoration design automation process and system automatically. As part of the integrated workflow, some embodiments in the present disclosure can include a computer-implemented method and/or a system for designing a dental restoration associated with a dental model of dentition. The method and/or system can in some embodiments provide a simplified, automated workflow to virtually design the dental restoration automatically and provide a dentist with feedback regarding preparation of the preparation tooth. In some embodiments, the integrated workflow along with any corresponding computer-implemented method and/or system can be implemented in a cloud computing environment. As is known in the art, the cloud computing environment can include, without limitation, one or more devices such as, for example one or more computing units such as servers, for example, networks, storage, and/or applications that are enabled over the internet and accessible to one or more permitted client devices. One example of a cloud computing environment can include Amazon Web Services, for example. Other cloud computing environment types can be used, including, but not limited to, private cloud computing environments available to a limited set of clients such as those within a company, for example. In some embodiments, the system can be implemented in a cloud computing environment.

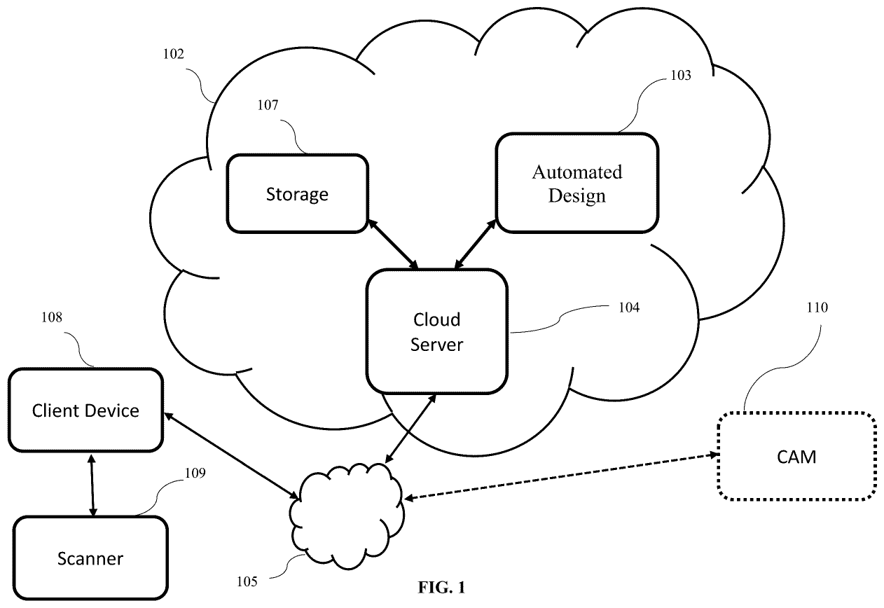

FIG. 1 illustrates one example of a cloud computing environment or system 102 for supporting integrated digital workflow for providing dental restoration design and/or fabrication according to some embodiments. The cloud computing environment 102 can include a dental restoration cloud server 104, automated design feature 103, storage 107 (direct storage in a storage device's file system, in a database, or in any other storage known in the art), and other components commonly known in the art for cloud computing in some embodiments. Each of the components in the cloud computing environment 102 can in some embodiments be interconnected for example by the dental restoration cloud server 104 or directly to one another or through one or more other servers or computers within the cloud computing environment or system 102. One or more client devices 108 can connect to the dental restoration cloud server 104 directly or through one or more networks 105 in some embodiments. The one or more client devices 108 can each connect with one or more scanners 109 known in the art to scan patient's dentition or a dental impression, for example, and provide a 3D virtual dental model of at least a portion of a patient's dentition. In some embodiments, the one or more client devices 108 can be located in a dental office, for example. The dental restoration cloud server 104 can connect to one or more fabrication providers 110 directly and/or through one or more networks 105. Only one dental restoration cloud server 104, one automated design feature 103, one client device 108, one scanner 109, one third party fabrication provider 110, and one storage 107 are shown in FIG. 1 in order to simplify and clarify the description. Embodiments of the cloud computing environment 102 can have multiple dental restoration cloud servers 104, automated design features 103, client devices 108, scanners 109, fabrication providers 110, and storage 107. Likewise, the features and arrangements/connections made by the various entities of FIG. 1 may differ in different embodiments. In some embodiments, the scanner 109 can be located in a dentist's office. In some embodiments, the client device 108 can be located in a dentist's office. In some embodiments the one or more fabrication providers 110 can include one or more dental laboratories, for example. One or more of the features illustrated in FIG. 1 can be implemented with and/or in one or more computing environments.

As part of dental restoration automation, the dental restoration cloud server 104 can in some embodiments receive dental restoration cases from the client devices 108 operated by clients, manage the dental restoration cases between the different clients, and, in turn, provide finished dental restoration designs and/or milled dental restorations to the clients. In some embodiments, the dental restoration cases may include design only cases that only request the dental restoration cloud server 104 to provide a virtual design of the dental restoration. In some embodiments, the dental restoration cases may request the dental restoration cloud server 104 not only to provide a design, but also to fabricate the dental restoration. In some embodiments, the dental restoration cases may request fabrication only.

Some embodiments of the computer-implemented method of virtual dental restoration design automation can include receiving a 3D virtual dental model of at least a portion of a patient's dentition. The 3D virtual dental model can include at least one virtual preparation tooth. The virtual preparation tooth can be a virtual representation of a physical preparation tooth prepared by a dentist. In some embodiments, the virtual dental restoration design automation can also receive an opposing 3D virtual dental model. The opposing 3D virtual dental model can include at least a portion of the patient's dentition opposite the physical preparation tooth. The opposing 3D virtual dental model can include at least one virtual opposing tooth corresponding the at least one virtual preparation tooth, but on the opposing jaw. In some embodiments the dentist can be a chair-side dentist located in a dental office. In some embodiments receiving the 3D virtual dental model can be performed in a cloud-computing environment.

In some embodiments, the 3D virtual dental model and opposing 3D virtual dental model can be generated by any process that scans a patient's dentition or a physical impression of the patient's dentition and generates a virtual 3D dental model of the patient's dentition. In some embodiments the 3D virtual dental model and opposing 3D virtual dental model can be generated from an intraoral scan of a patient's dentition. In some embodiments, for example, the intraoral scans can be performed to produce two virtual dental models—a 3D virtual dental model with the virtual preparation tooth, and an opposing 3D virtual dental model with the virtual opposing tooth. The intraoral scanning device can, for example, be handheld in some embodiments, and can be used by a dentist, technician, or user to scan a patient's dentition. The standard intraoral scanning device and associated hardware and software can then generate the virtual 3D dental model as a standard STL file or other suitable standard format. One example of an intraoral scanner can be an Itero© intraoral scanner provided by Align Technologies. Another example of an intraoral scanner can be the i700 intraoral scanner provided by Medit. Other intraoral scanners or other scanners and/or scanning techniques for producing a 3D virtual dental model of at least a portion of the patient's dentition can also be used. In some embodiments, the scanning process can produce STL, PLY, or CTM files, for example that can be suitable for use with a dental design software, such as FastDesign™ dental design software provided by Glidewell Laboratories of Newport Beach, Calif. In some embodiments, the intraoral scan can be performed outside of a dental laboratory. For example, in some embodiments the intraoral scan can be performed in a dental office. In some embodiments the dentist/dental office can be part of a Dental Support Organization (“DSO”). The DSO can include one or more dentists practicing together.

In some embodiments the one or more steps disclosed herein can occur in near real time. For example, in some embodiments, one or more steps in the present disclosure can be performed while a patient visits a dental office for treatment. In many cases, the patient may be under general or local anesthesia and in the treatment chair while the dentist prepares the one or more physical preparation teeth and then scans at least a portion of the patient's dentition to capture the prepared physical tooth and portion of patient's dentition to generate the 3D virtual dental model. In some embodiments, the dentist can also scan at least a portion of the patient's opposing teeth to generate the opposing 3D virtual dental model of the opposing teeth/jaw while the patient is in the treatment chair. The scanning can be performed using an intraoral scanner or other scanner in the dentist's office while the patient is still in the chair receiving treatment.

Once the 3D virtual dental model that includes one or more virtual preparation teeth and the opposing 3D virtual dental model are generated, the dentist, staff, or any other authorized user at the dental office can create a virtual case for the patient as part of the virtual dental restoration design automation, and upload the virtual dental models (also known as “scans”), which can be received for virtual restoration automated design. In some embodiments, the automated design can be performed in a cloud-computing environment (“the cloud”). The virtual case can be stored or associated with information regarding a particular patient and treatment together on a storage device or a database, for example. In some embodiments, the information can include, for example, information regarding the patient and preparation, such as (but not limited to) a patient name, a user-selectable preparation tooth number, a user-selectable material, a user-selectable preparation tooth shade and the 3D virtual dental model itself.

In some embodiments, the 3D virtual dental model and opposing 3D virtual dental model can be provided to the automated dental restoration design process and system through a graphical user interface (“GUI”) that can be displayed on a client device by the cloud computing environment. In some embodiments, for example, the GUI can provide an interface that allows a client to log into a dental restoration design server and upload the virtual 3D dental model scanned. In some embodiments, the client can be a dentist, dental technician, or any other user, for example. In some embodiments the client can be located in a dental office.

In some embodiments, the cloud computing environment can receive the 3D virtual dental model from a client device that can include, for example, a computing device in a dental office. The case generation and uploading can be performed through the interface, such as a Graphical User Interface (“GUI”) displayed on the client device display to allow input of the case information and upload of the 3D virtual dental model. In some embodiments, the interface can connect to one or more clouds, for example, or to one or more computer servers or other systems run by a dental laboratory and connected to the dental office through the Internet to store the case information. In some embodiments, the case can be created and information provided manually by the dentist or others at the dental office, or automatically by the scanning software used by the scanner in some embodiments. For example, in some embodiments upon scanning the patient's dentition at the dental office, the Itero® intraoral scanner can automatically open a case and populate the case information, and upload the 3D virtual dental model.

Some embodiments of the computer-implemented method of virtual dental restoration design automation can include performing an automated virtual restoration design using the 3D virtual dental model. In some embodiments, the automated virtual restoration design can be performed during a patient visit to a dental office while the patient is in the chair. In some embodiments, the automated virtual restoration design can be performed in a cloud-computing environment.

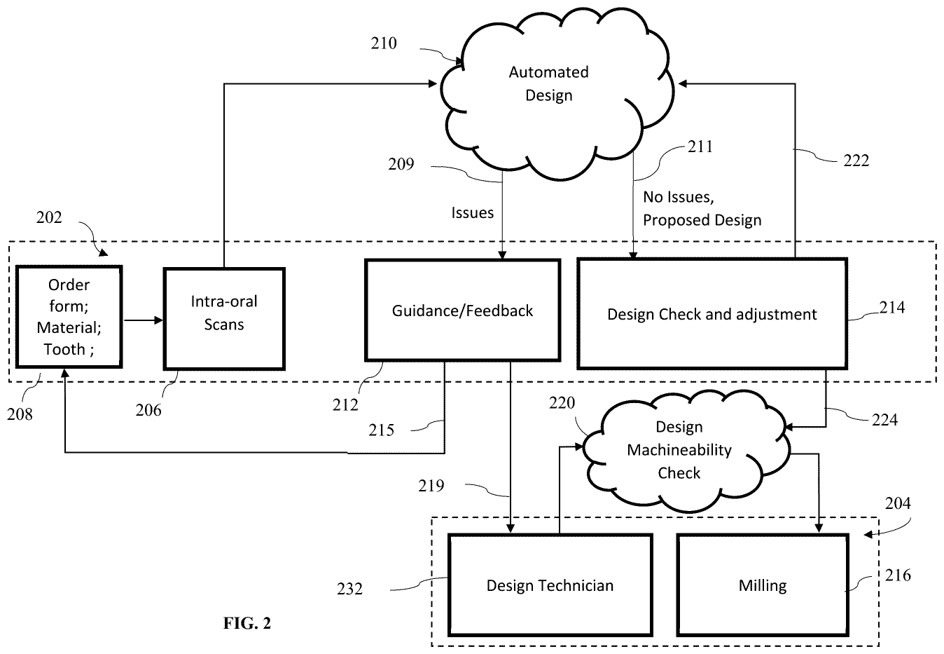

FIG. 2 is a diagram illustrating an overview of one example in some embodiments, for example. A dentist or dental technician or other user located in a dental office 202 can open a new case 208 and use an intraoral scanner 206 to scan at least a portion of a patient's dentition that includes at least one physical preparation tooth in some embodiments associated with the case 208. Another intraoral scan of at least a portion of the patient's opposing dentition (i.e. the other jaw) can also be taken in some embodiments and also associated with the same case 208. The intraoral scanner can generate a virtual dental model for each scan. For example, the intraoral scanner can generate a 3D virtual dental model for the dentition with the at least one physical preparation tooth and generate an opposing 3D virtual dental model for the opposing dentition. In some embodiments, the intraoral scanning 206 can be performed first and automatically generate the order form 208. Once the intraoral scanning is complete, the 3D virtual dental model and the opposing 3D virtual dental model can be sent to automated design 210 in some embodiments.

The automated design 210 can be performed in a cloud-computing environment in some embodiments. The automated design 210 can in some embodiments, determine a quality of the physical preparation tooth prepared by the dentist. If the physical preparation tooth is of an acceptable quality and no issues are found at 211, the automated design 210 can generate a virtual restoration and display the generated virtual restoration on a computing device display to the dentist, dental technician, or other user in the dental office 202 for a design check (“DC”) 214 in some embodiments. The design check 214 can allow the dentist, etc. to make adjustments to the generated virtual restoration and/or a margin line in some embodiments.

In some embodiments, the dentist, other user, etc. can make minor adjustments which can be applied to the virtual restoration. In some embodiments, the dentist, other user, etc. can make major adjustments. In some embodiments, minor and major changes can be applied as they are made. Major adjustments can include, but are not limited to, adjusting the margin line, for example. In some embodiments, major adjustments 222 trigger automated design 210 as they are made to regenerate a new virtual restoration based on the major adjustments made. Once all adjustments are complete and the design is finalized by the dentist or user, the final virtual restoration design is forwarded 224 to design machinability check 220. In some embodiments, the dentist, other user, etc. can simply accept the original proposed virtual restoration without making any adjustments, which is then forwarded 224 to the design machinability check 220.

The design machinability check 220 can determine whether the generated virtual restoration can be auto-milled or not. The design machinability check 220 can also be located in a cloud-computing environment. The cloud-computing environment can be the same for both the automated design 210 and the design machinability check 220, or separate cloud-computing environments can be used. If the virtual restoration can be auto-milled, it can be sent to milling 216 for physical fabrication of the virtual restoration. In some embodiments, the milling 216 can be auto-milling if the design machinability check 220 is passed. One example of automated milling can be found in U.S. Pat. No. 10,470,853B2 to Leeson et al., the entirety of which is hereby incorporated by reference. In some embodiments, the milling 216 can be manual milling if the design machinability check 220 indicates auto-milling cannot be performed. In some embodiments, the milling 216 can occur at a dental laboratory 204.

In some embodiments, if the automated design 210 identifies one or more issues 209 with the physical preparation tooth, the computer-implemented method can provide virtual guidance/feedback at 212 to the dentist and/or other user, etc. in the dental office 202. In some embodiments, the dentist can then adjust the physical preparation tooth, the physical opposing tooth, and/or a portion of the patent's dentition based on the virtual guidance/feedback and rescan the patient's adjusted physical dentition that can include one or more physical preparation teeth and opposing tooth/teeth and/or dentition 215. The dentist or user can then re-upload the rescanned 3D virtual models of the rescanned 3D virtual dental model that can include at least one rescanned virtual preparation tooth and/or at least one corresponding rescanned opposing tooth in some embodiments. The rescans can be added to the same order form 208 in some embodiments, and processing can resume as described in FIG. 2.

In some embodiments, the dentist/user can, upon receiving virtual guidance feedback 212 indicating one or more issues, simply accept and submit 219 the 3D virtual dental model and any opposing 3D virtual dental model to a design lab technician 232 or other user who is typically with a dental laboratory 204. The design lab technician or other user can then determine one or more reduction regions on the virtual preparation tooth based on the issues raised from automated design to design a virtual reduction coping as described in U.S. Pat. No. 11,351,015B2 to Leeson, et al. (hereafter, '015). The design lab technician can then fabricate a physical reduction/guidance coping as described in '015, and send the physical reduction/guidance coping to the dentist to provide physical feedback to the dentist or user at the dental office regarding which physical preparation tooth, opposing tooth, and/or surrounding dentition areas to physically reduce. In some embodiments, the design technician can also design a virtual restoration for the virtual reduced preparation tooth and send the designed virtual restoration to design machinability check 220 for subsequent fabrication of a physical restoration. In some embodiments, the design lab technician can use automated design 210 to generate the virtual restoration from a virtual reduced preparation tooth, virtual reduced opposing tooth, and/or virtual reduced patient dentition. In some embodiments, the dental technician can manually design the virtual restoration.

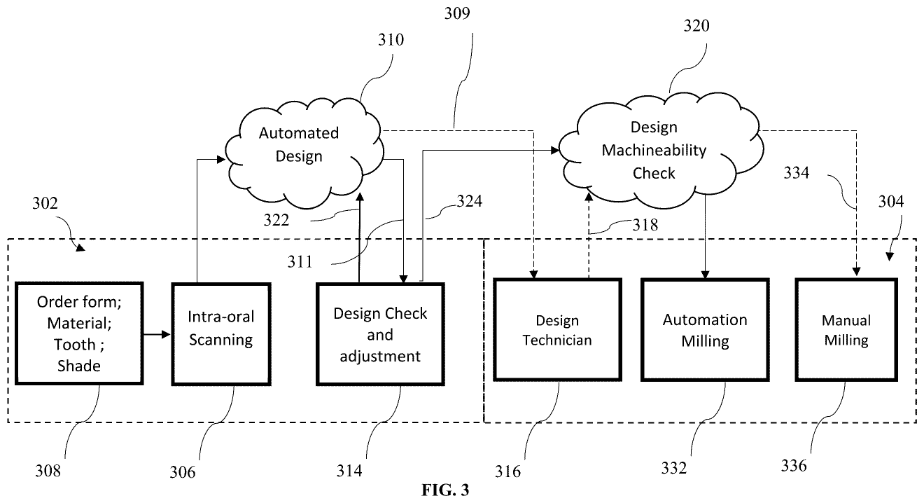

FIG. 3 is a diagram illustrating an overview of one example of dental restoration automation in some embodiments. A dentist or dental technician or other user located in a dental office 302 can open a new case 308 and use an intraoral scanner 306 to scan at least a portion of a patient's dentition that includes at least one physical preparation tooth in some embodiments associated with the case 308. Another intraoral scan of at least a portion of the patient's opposing dentition (i.e. the other jaw) can also be taken in some embodiments and also associated with the same case 308. The intraoral scanner can generate a virtual dental model for each scan. For example, the intraoral scanner can generate a 3D virtual dental model for the dentition with the at least one physical preparation tooth and generate an opposing 3D virtual dental model for the opposing dentition. In some embodiments, the intraoral scanning 306 can be performed first and automatically generate the order form 308. Once the intraoral scanning is complete, the 3D virtual dental model and the opposing 3D virtual dental model can be sent to automated design 310 in some embodiments.

The automated design 310 can be performed in a cloud-computing environment in some embodiments. The automated design 310 can in some embodiments, determine a quality of the physical preparation tooth prepared by the dentist. If the physical preparation tooth is of an acceptable quality and no issues are found at 311, the automated design 310 can generate a virtual restoration and display the generated virtual restoration on a computing device display to the dentist, dental technician, or other user in the dental office 302 for a design check (“DC”) 314 in some embodiments. The design check 314 can allow the dentist, etc. to make adjustments to the generated virtual restoration and/or a margin line in some embodiments as discussed previously with respect to FIG. 2.

In some embodiments, the dentist, other user, etc. can make minor adjustments which can be applied to the virtual restoration. In some embodiments, the dentist, other user, etc. can make major adjustments. Major adjustments can include, but are not limited to, adjusting the margin line, for example. In some embodiments, major adjustments 322 trigger automated design 310 as they are made to regenerate a new virtual restoration based on the major adjustments made. In some embodiments, minor and major changes can be applied as they are made. Once all adjustments are complete and the design is finalized by the dentist or user, the final virtual restoration design is forwarded 324 to design machinability check 320. In some embodiments, the dentist, other user, etc. can simply accept the original proposed virtual restoration without making any adjustments, which is then forwarded 324 to the design machinability check 320.

In some embodiments, if the automated design 310 identifies one or more issues 309 with the physical preparation tooth, the computer-implemented method can forward the uploaded 3D virtual dental model with the one or more virtual preparation teeth and the opposing tooth/dentition 3D virtual dental model 309 to a design technician 316. In some embodiments, the design technician 316 can be with a dental laboratory 304, for example. In some embodiments, upon completion of the manual design, the dental technician 316 can forward a manually generated virtual restoration to the design machinability check 320 and fabrication processing as discussed previously. In some embodiments, the dental laboratory 304 can also provide feedback to the dentist and/or user regarding the one or more issues with the physical preparation tooth the automated design 310 determined. In some embodiments, the feedback can include on or more images or 3D models with any issues marked. In some embodiments, the design technician 316 can, upon completing the virtual restoration design, provide at 318 the virtual restoration design for design machinability check 320.

The design machinability check 320 can determine whether the generated virtual restoration can be auto-milled or not. The design machinability check 320 can also be located in a cloud-computing environment. The cloud-computing environment can be the same for both the automated design 310 and the design machinability check 320, or separate cloud-computing environments can be used. If the virtual restoration can be auto-milled, it can be sent to automatic milling 332 for physical fabrication of the virtual restoration. In some embodiments, the milling 316 can be auto-milling if the design machinability check 320 is passed. One example of automated milling can be found in U.S. Pat. No. 10,470,853B2 to Leeson et al., the entirety of which is hereby incorporated by reference. In some embodiments, the milling 316 can be manual milling 336 if the design machinability check 320 indicates auto-milling cannot be performed.

The virtual restoration can be sent 334 to manual milling 336 in some embodiments. In some embodiments, the milling can occur at a dental laboratory 304.

In some embodiments, performing the automated virtual restoration design can include, for example, one or more of the following steps: decimation of the 3D virtual dental model, meshing, segmentation, determining an occlusal direction, determining a bite alignment, preparation die localization, determining a buccal direction, determining a margin for the at least one virtual preparation tooth, determining an insertion direction onto the virtual preparation tooth, determining cement space for the virtual preparation tooth, generating the virtual restoration, and pulling the virtual restoration to the margin. In some embodiments, one or more of the automated virtual restoration design steps can be performed on the 3D virtual model that includes the one or more virtual preparation tooth/teeth. In some embodiments, one or more of the automated virtual restoration design steps can be performed on the opposing 3D virtual model that includes one or more opposing virtual tooth/teeth opposite the corresponding or more virtual preparation teeth. In some embodiments, the one or more steps can be performed sequentially or in any other suitable order. In some embodiments, one or more steps can be excluded.

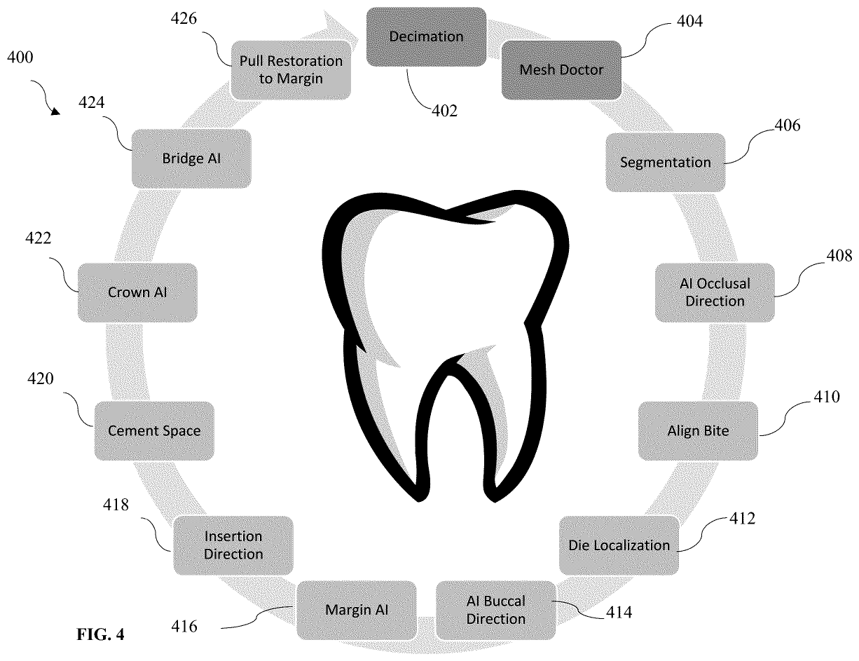

FIG. 4 illustrates one example of one or more of the automated virtual restoration design steps 400. The one or more automated virtual restoration design steps can be performed in a cloud-computing environment in some embodiments. In some embodiments, performing the automated virtual restoration design can include, for example, one or more of the following steps can be performed sequentially or in any other suitable order: decimation of the 3D virtual dental model 402, mesh repairs 404, segmentation 406, determining an occlusal direction 408, determining a bite alignment 410, preparation die localization 412, determining a buccal direction 414, determining a margin for the at least one virtual preparation tooth 416, determining an insertion direction onto the virtual preparation tooth 418, determining cement space for the virtual preparation tooth 420, generating the virtual restoration 422 or bridge 424, and pulling the virtual restoration to the margin 426. In some embodiments, one or more steps can be performed in any order. In some embodiments, one or more steps can be performed sequentially in the order shown in FIG. 4, for example. In some embodiments, some steps can be excluded.

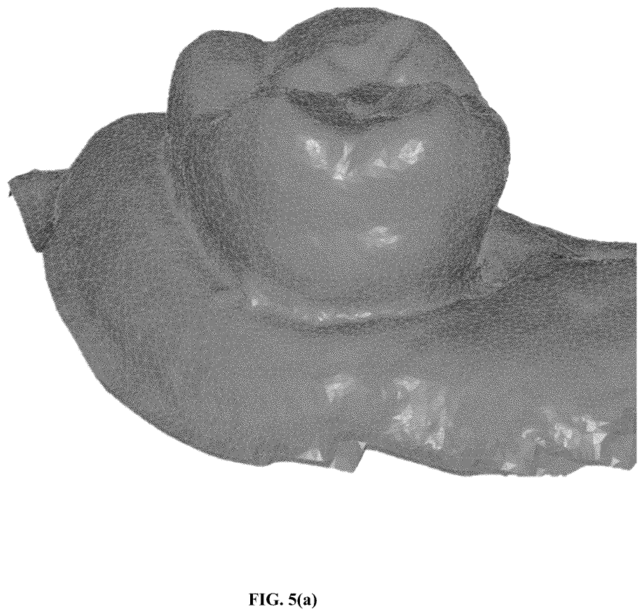

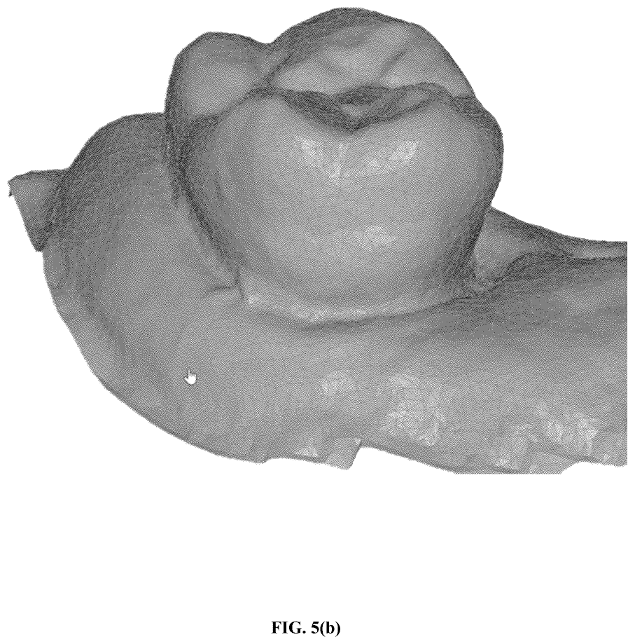

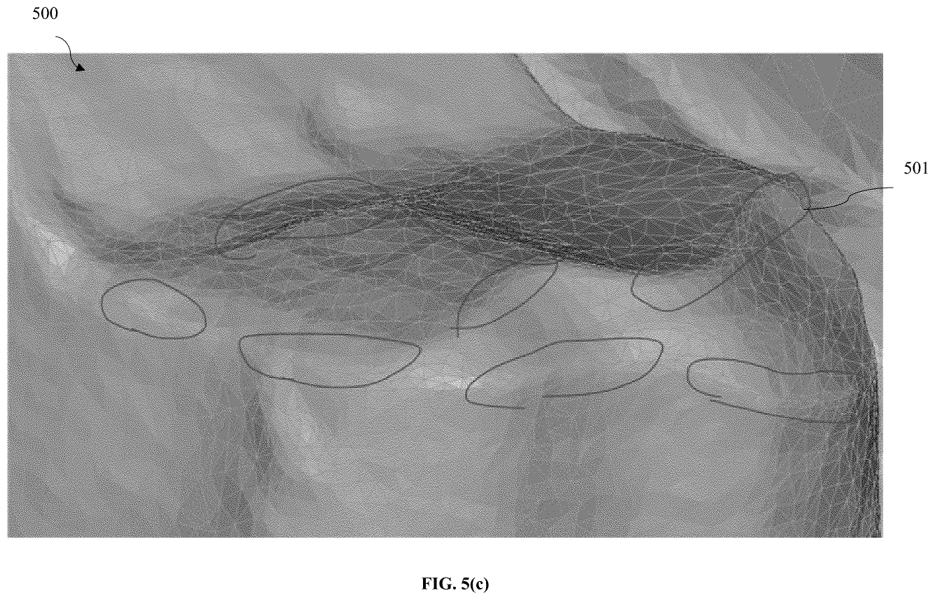

In some embodiments, the automated virtual restoration design can include optionally performing decimation. Decimation can include reducing a file size of the 3D virtual dental model. This can be achieved in some embodiments by reducing the number of polygons in the 3D virtual dental model. In some embodiments, the amount of decimation to perform can be a user-configurable value that can, for example, sample a subset of polygons or points in the 3D virtual dental model. In some embodiments, decimation can be performed by selecting a subset of points defining the 3D virtual dental model. For example, in some embodiments, the computer-implemented method can select every 2^{nd} or 3^{rd} point in the 3D virtual dental model. In some embodiments, decimation can be performed while the patient is in the dental office. In some embodiments, decimation can be triggered when the number polygons (virtual triangles in some embodiments) in a virtual mesh exceed a user-configurable threshold value. In some embodiments, decimation can include combining two or more virtual triangles in the virtual mesh to reduce the number of virtual triangles in the mesh, adjusting the mesh to increase uniformity, and decreasing virtual triangle size in areas of greater curvature. FIG. 5(a) illustrates an example of an initial virtual mesh having more virtual triangles than the user-configurable threshold for decimation. FIG. 5(b) illustrates an example of a decimated virtual mesh having fewer virtual triangles than the user-configurable threshold for decimation. FIG. 5(c) illustrates an example of a portion of a virtual mesh 500 with one or more regions of increased curvature having smaller virtual triangles 501 than other less curved regions. Other highly curved regions with smaller virtual triangles are also circled in FIG. 5(c).

In some embodiments, the automated virtual restoration design can optionally include performing mesh repairs. In some embodiments, mesh repairs can include removing triangles, hole filling, and smoothing out the virtual dental model, for example. In some embodiments, mesh repairs can be performed while the patient is in the dental office. In some embodiments, mesh repairs can include correcting triangulation of the mesh (grid), removing any self intersection of the mesh, removing spikes and long-thin virtual triangles, removing tunnels, filling holes, removing empty points (not belonging to any virtual triangles), and removing separate small mesh regions. In some embodiments, mesh repairs can be performed during treatment of a patient in a dental office.

In some embodiments, the automated virtual restoration design can include automatically performing segmentation on the 3D virtual dental model. Segmentation can identify or more virtual teeth, gum, and/or other features within the 3D virtual dental model. One or more examples of automatically performing segmentation on the 3D virtual dental model can be found in U.S. application Ser. No. 17/140,739 of Azernikov et al., the entirety of which is hereby incorporated by reference. As described in that application, segmentation can include, for example, receiving a 3D virtual model of patient scan data of at least a portion of a patient's dentition; generating a panoramic image from the 3D virtual model; labeling, using a first trained neural network, one or more regions of the panoramic image to provide a labeled panoramic image; mapping one or more regions of the labeled panoramic image to one or more corresponding coarse virtual surface triangle labels in the 3D virtual model to provide a labeled 3D virtual model; and segmenting the labeled 3D virtual model to provide a segmented 3D virtual model. Another example of automatically performing segmentation on the 3D virtual dental model also can be found in U.S. application Ser. No. 16/451,968 to Nikolskiy et al., the entirety of which is hereby incorporated by reference. In some embodiments, segmentation can be performed during treatment of a patient while the patient is in the dental office.

In some embodiments, one or more features can be automatically determined using a trained 3D deep neural network (“DNN”) on the volumetric (voxel) representation. In some embodiments, the DNN can be a convolutional neural network (“CNN”), which is a network that uses convolution in place of the general matrix multiplication in at least one of the hidden layers of the deep neural network. A convolution layer can calculate its output values by applying a kernel function to a subset of values of a previous layer. The computer-implemented method can train the CNN by adjusting weights of the kernel function based on the training data. The same kernel function can be used to calculate each value in a particular convolution layer.

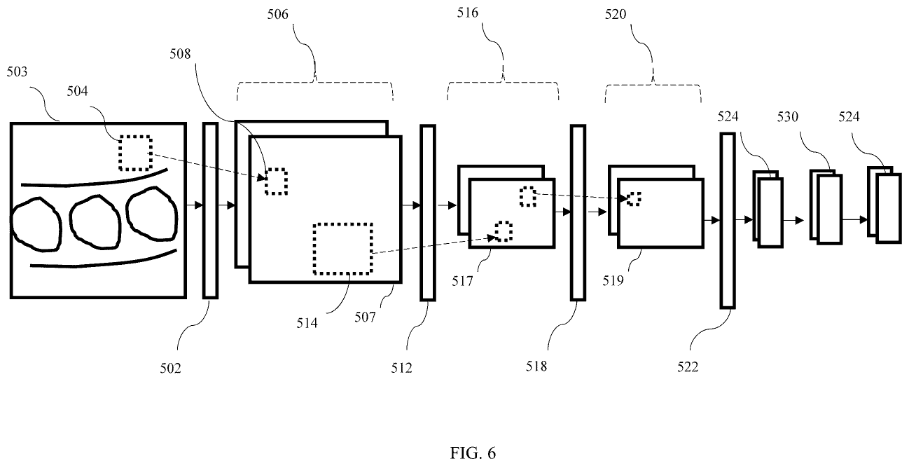

FIG. 6 illustrates an example of a CNN in some embodiments. For illustration purposes, a 2D CNN is shown. A 3D CNN can have a similar architecture, but use a three dimensional kernel (x-y-z axis) to provide a three dimensional output after each convolution. The CNN can include one or more convolution layers, such as first convolution layer 502. The first convolution layer 502 can apply a kernel (also referred to as a filter) such as kernel 504 across an input image such as input image 503 and optionally apply an activation function to generate one or more convolution outputs such as first kernel output 508. The first convolution layer 502 can include one or more feature channels. The application of the kernel such as kernel 504 and optionally an activation function can produce a first convoluted output such as convoluted output 506. The kernel can then advance to the next set of pixels in the input image 503 based on a stride length and apply the kernel 504 and optionally an activation function to produce a second kernel output. The kernel can be advanced in this manner until it has been applied to all pixels in the input image 503. In this manner, the CNN can generate a first convoluted image 506, which can include one or more feature channels. The first convoluted image 506 can include one or more feature channels such as 507 in some embodiments. In some cases, the activation function can be, for example, a RELU activation function. Other types of activation functions can also be used.

The CNN can also include one or more pooling layers such as first pooling layer 512. First pooling layer can apply a filter such as pooling filter 514, to the first convoluted image 506. Any type of filter can be used. For example, the filter can be a max filter (outputting the maximum value of the pixels over which the filter is applied) or an average filter (outputting the average value of the pixels over which the filter is applied). The one or more pooling layer(s) can down sample and reduce the size of the input matrix. For example, first pooling layer 512 can reduce/down sample first convoluted image 506 by applying first pooling filter 514 to provide first pooled image 516. The first pooled image 516 can include one or more feature channels 517. The CNN can optionally apply one or more additional convolution layers (and activation functions) and pooling layers. For example, the CNN can apply a second convolution layer 518 and optionally an activation function to output a second convoluted image 520 that can include one or more feature channels 519. A second pooling layer 522 can apply a pooling filter to the second convoluted image 520 to generate a second pooled image 524 that can include one or more feature channels. The CNN can include one or more convolution layers (and activation functions) and one or more corresponding pooling layers. The output of the CNN can be optionally sent to a fully connected layer, which can be part of one or more fully connected layers 530. The one or more fully connected layers can provide an output prediction such as output prediction 524. In some embodiments, the output prediction 524 can include labels of teeth and surrounding tissue, for example. In some embodiments, the output prediction 524 can include identification of one or more features in the 3D digital dental model.

In some embodiments, the automated virtual restoration design can include automatically determining an occlusal direction. One example of automatically determining an occlusal direction can be found in U.S. application Ser. No. 17/245,944 of Azernikov et al., the entirety of which is hereby incorporated by reference. In some embodiments, determining the occlusal direction can be performed while the patient is in the dental office.

In some embodiments, the occlusal direction can be determined automatically using a occlusal direction trained neural network. In some embodiments, the occlusal direction trained CNN can be a 3D CNN trained using one or more 3D voxel representations, each representing a patient's dentition, optionally with augmented data such as surface normal for each voxel. 3D CNNs can perform 3D convolutions, which use a 3D kernel instead of a 2D kernel, and operate on 3D input. In some embodiments, the trained 3D CNN receives 3D voxel representations with voxel normals. In some embodiments, a N×N×N×3 float tensor can be used. In some embodiments, N can be 100, for example. Other suitable values of N can be used. In some embodiments, the trained 3D CNN can include 4 levels of 3D convolutions and can include 2 linear layers. In some embodiments, the 3D CNN can operate in the regression regime in which it regresses voxel and their corresponding normals representing of a patient's dention to three numbers, X, Y, Z, coordinates of the unit occlusal vector. In some embodiments, a training set for the 3D CNN can include one or more 3D voxel representations, each representing a patient's dentition. In some embodiments, each 3D voxel representation in the training set can include an occlusal direction marked manually by a user or by other techniques known in the art. In some embodiments, the training set can include tens of thousands of 3D voxel representations, each with a marked occlusion direction. In some embodiments, the training dataset can include 3D point cloud models with marked occlusion direction in each 3D point cloud model. Accordingly, one occlusal direction for each image/model (3D voxel representation) of a patient's dentition is marked in the training dataset by a technician, and the training dataset can include tens of thousands of images/models (3D voxel representations) of corresponding patient dentition. In the training data, coordinates of the unit occlusal vector can be such that X\hat{ }2+Y\hat{ }2+Z\hat{ }2=1 in some embodiments for example.

In some embodiments, the automated virtual restoration design can include automatically determining a bite setting. In some embodiments, the bite setting can be determined between the 3D virtual dental model that includes the one or more virtual preparation tooth/teeth and the opposing 3D virtual dental model that includes the one or more corresponding virtual opposing tooth/teeth. In some embodiments, automatically determining a bite setting on the 3D virtual dental model can be found in U.S. application Ser. No. 17/007,922 of Chelnokov et al., the entirety of which is hereby incorporated by reference. As described in that application, in some embodiments, automatically determining the bite setting can include receiving first and second virtual jaw models such as the 3D virtual dental model with the virtual preparation tooth/teeth and the opposing 3D virtual dental model with the corresponding virtual opposing tooth/teeth, determining a rough bite approximation of the first and second virtual jaw models, determining one or more initial bite positions of the first and second virtual jaw models from the rough approximation, determining one or more iterative bite positions of the first and second virtual jaw models for each of the one or more initial bite positions, determining a score for each iterative bite position, and outputting the bite setting based on the score. In some embodiments, determining the bite setting can be performed while the patient is in the dental office.

In some embodiments, the automated virtual restoration design can include automatically performing preparation die localization. One example of automatically performing die localization can be found in U.S. application Ser. No. 17/245,944 of Azernikov et al., which was previously incorporated by reference. As described in that application, automatically performing die localization can include determining the 3D center of the virtual preparation using a neural network on an occlusally aligned 3D point cloud. In some embodiments, preparation die localization can be performed while the patient is in the dental office.

In some embodiments, the 3D center of the digital preparation die can be determined automatically. For example, in some embodiments, the 3D center of the digital preparation can be determined using a neural network on an occlusally aligned 3D point cloud. In some embodiments, the trained neural network can provide a 3D coordinate of a center of digital preparation bounding box. In some embodiments, the neural network can be any neural network that can perform segmentation on a 3D point cloud. For example, in some embodiments, the neural network can be a PointNet++ neural network segmentation as described in the present disclosure. In some embodiments, the digital preparation die can be determined by a sphere of a fixed radius around the 3D center of the digital preparation. In some embodiments, the fixed radius can be 0.8 cm for molar and premolars, for example. Other suitable values for the fixed radius can be determined and used in some embodiments, for example. In some embodiments, training the neural network can include using the sampled point cloud (without augmentation) of the digital jaw, centered in the center of mass of the jaw. In some embodiments, the digital jaw point cloud can be oriented in such a way that the occlusal direction is positioned vertically. In some embodiments, the computer-implemented method can train a neural network to determine the digital preparation site/die in a 3D digital dental model by using a training dataset that can include 3D digital models of point clouds of a patient's dentition such as a digital jaw that can include a preparation site, with one or more points within the margin line of the preparation site marked by user using an input device, or any technique known in the art. In some embodiments, the training set can be in the tens of thousands. In some embodiments, the neural network can in operation utilize segmentation to return a bounding box containing the selected points. In some embodiments, the segmentation used can be PointNet++ segmentation, for example.

In some embodiments, the 3D center of the digital preparation die can be determined automatically based on a flat depth map image of the jaw. In the training dataset, the position of a die center can be determined as a geometrical center of margin marked by technicians. In some embodiments, final margin points from completed cases can be used, for example. In some embodiments, the network can receive a depth map image of a jaw from occlusal view and return a position (X, Y) of a die center in the pixel coordinates of the image. For training, a dataset that contains depth map images and corresponding correct answer—float X and Y values—can be used. In some embodiments, the training set can be in the tens of thousands.

In some embodiments, the 3D center of the digital preparation die can optionally be set manually by a user. In some embodiments, the 3D center of the digital preparation die can be set using any technique known in the art.

In some embodiments, the automated virtual restoration design can include automatically determining a buccal direction. In some embodiments, the 3D digital model can include a buccal direction. In some embodiments, the buccal direction can be set manually by a user. In some embodiments, the buccal direction can be determined using any technique known in the art. One example of automatically determining a buccal direction can be found in U.S. application Ser. No. 17/245,944, which was previously incorporated by reference. In some embodiments, automatically determining a buccal direction can include, for example, providing a 2D depth map image of the 3D virtual model mesh to a trained 2D convolutional neural network (“CNN”) such as GoogleNet Inception v3. In some embodiments, the trained 2D CNN operates on the image representation. In some embodiments, determining the buccal direction can be performed while the patient is in the dental office.

In some embodiments, a trained 2D CNN operates on the image representation. In some embodiments, the buccal direction can be determined by providing a 2D depth map image of the 3D digital model mesh to a trained 2D CNN. In some embodiments, the method can optionally include generating a 2D image from the 3D digital model. In some embodiments, the 2D image can be a 2D depth map. The 2D depth map can include a 2D image that contains in each pixel a distance from an orthographic camera to an object along a line passing through the pixel. The object can be, for example, a digital jaw model surface, in some embodiments, for example. In some embodiments, an input can include, for example, an object such as a 3D digital model of patient's dentition (“digital model”), such as a jaw, and a camera orientation. In some embodiments, the camera orientation can be determined based on an occlusion direction. The occlusal direction is a normal to an occlusal plane and the occlusal plane can be determined for the digital model using any technique known in the art. Alternatively, in some embodiments, the occlusal direction can be specified by a user using an input device such as a mouse or touch screen to manipulate the digital model on a display, for example, as described herein. In some embodiments, the occlusal direction can be determined, for example, using the Occlusion Axis techniques described in U.S. patent application Ser. No. 16/451,968 (U.S. Patent Publication No. US20200405464A1), of Nikolskiy et al., the entirety of which is incorporated by reference herein.

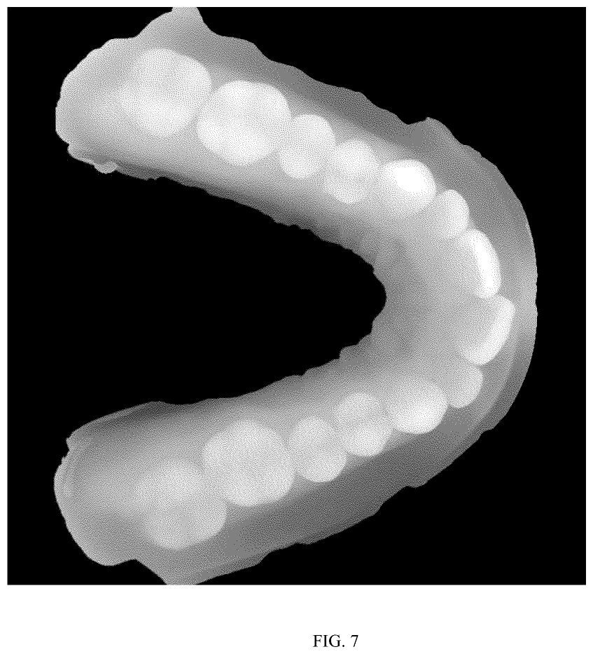

The 2D depth map can be generated using any technique known in the art, including, for example z-buffer or ray tracing. For example, in some embodiments, the computer-implemented method can initialize the depth of each pixel (j, k) to a maximum length and a pixel color to a background color, for example. The computer-implemented method can for each pixel in a polygon's projection onto a digital surface such as a 3D digital model determine a depth, z of the polygon at (x, y) corresponding to pixel (j, k). If z<depth of pixel (j, k), then set the depth of the pixel to the depth, z. “Z” can refer to a convention that the central axis of view of a camera is in the direction of the camera's z-axis, and not necessarily to the absolute z axis of a scene. In some embodiments, the computer-implemented method can also set a pixel color to something other than a background color for example. In some embodiments, the polygon can be a digital triangle, for example. In some embodiments, the depth in the map can be per pixel. FIG. 7 illustrates an example of a 2D depth map of a digital model in some embodiments.

In some embodiments, 2D depth map image can include a Von Mises average of 16 rotated versions of the 2D depth map. In some embodiments, the buccal direction can be determined after determining occlusal direction and the 3D center of the digital preparation die. In some embodiments, the 2D depth map image can be of a portion of the digital jaw around the digital preparation die. In some embodiments, regression can be used to determine the buccal direction. In some embodiments, the 2D CNN can include GoogleNet Inception v3, known in the art, for example. In some embodiments, the computer-implemented method can train the buccal trained neural network using a training dataset. In some embodiments, the training dataset can include buccal directions marked in a 3D point cloud model, for example. In some embodiments, the training data set can include tens of thousands to hundreds of thousands of images. In some embodiments, the computer-implemented method pre-process the training dataset by converting each training image to a 2D depth-map as disclosed previously, and training the 2D CNN using the 2D depth-map, for example.

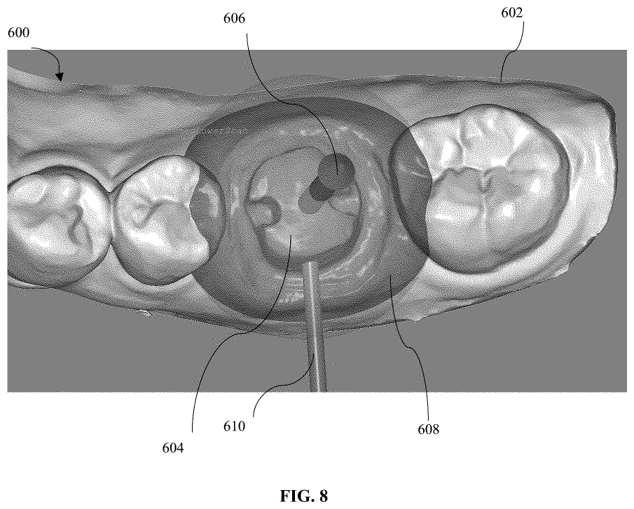

In some embodiments, the occlusion direction, the buccal direction, and/or the preparation die region can provide a normalized orientation of the 3D virtual model, for example. For example, the occlusal direction is a normal to an occlusal plane, the virtual preparation die can be a region around the virtual preparation tooth, and the buccal direction can be a direction toward the cheek in the mouth. FIG. 8 illustrates an example of a 3D virtual model 600 of at least a portion of a patient's dentition that can include, for example a virtual jaw 602 that includes a virtual preparation tooth 604. The 3D virtual model 600 can include an occlusion direction 606, a virtual preparation die region 608, and a buccal direction 610.

In some embodiments, the automated virtual restoration design can include automatically determining an adjustable margin line of the virtual preparation tooth. One example of automatically determining an adjustable margin line of the virtual preparation tooth can be found in U.S. application Ser. No. 17/245,944 of Azernikov et al., the entirety of which is hereby incorporated by reference.

In some embodiments, the computer-implemented method can determine the margin line proposal by receiving the 3D digital model having a digital preparation site and, using an inner representation trained neural network, determine an inner representation of the 3D digital model.

Some embodiments of the computer-implemented method can include determining, using an inner representation trained neural network, an inner representation of the 3D digital model. In some embodiments, the inner representation trained neural network can include an encoder neural network. In some embodiments, the inner representation trained neural network can include a neural network for 3D point cloud analysis. In some embodiments, the inner representation trained neural network can include a trained hierarchal neural network (“HNN”). In some embodiments, the HNN can include a PointNet++ neural network. In some embodiments, the HNN can be any message-passing neural network that operates on geometrical structures. In some embodiments, the geometrical structures can include graphs, meshes, and/or point clouds.

In some embodiments, the computer-implemented method can use an HNN such as PointNet++ for encoding. PointNet++ is described in “PointNet++: Deep Hierarchical Feature Learning on Point Sets in a Metric Space”, Charles R. Qi, Li Yi, Hao Su, Leonidas J. Guibas, Stanford University, June 2017, the entirety of which is hereby incorporated by reference. Hierarchal neural networks can, for example, process a sampled set of points in a metric space in a hierarchal way. An HNN such as PointNet++ or other HNN can be implemented by determining a local structure induced by a metric in some embodiments. In some embodiments, the HNN such as PointNet++ or other HNN can be implemented by first partitioning a set of points into two or more overlapping local regions based on the distance metric. The distance metric can be based on the underlying space. In some embodiments, the local features can be extracted. For example, in some embodiments, granular geometric structures from small local neighborhoods can be determined. The small local neighborhood features can be grouped into larger units in some embodiments. In some embodiments, the larger units can be processed to provide higher level features. In some embodiments, the process is repeated until all features of the entire point set are obtained. Unlike volumetric CNNs that scan the space with fixed strides, local receptive fields in HNNs such as PointNet++ or other HNN are dependent on both the input data and the metric. Also, in contrast to CNNs that scan the vector space agnostic of data distribution, the sampling strategy in HNNs such as PointNet++ or other HNN generates receptive fields in a data dependent manner.

In some embodiments, the HNN such as PointNet++ or other HNN can, for example, determine how to partition the point set as well as abstract sets of points or local features with a local feature learner. In some embodiments, the local feature learner can be PointNet, or any other suitable feature learner known in the art, for example. In some embodiments, the local feature learner can process a set of points that are unordered to perform semantic feature extraction, for example. The local feature learner can abstract one or more sets of local points/features into higher level representations. In some embodiments, the HNN can apply the local feature learner recursively. For example, in some embodiments, PointNet++ can apply PointNet recursively on a nested portioning of an input set.

In some embodiments, the HNN can define partitions of a point set that overlap by defining each partition as a neighborhood ball in Euclidean space with parameters that can include, for example, a centroid location and a scale. The centroids can be selected from the input set by farthest point sampling known in the art, for example. One advantage of using an HNN can include, for example, efficiency and effectiveness since local receptive fields can be dependent on input data and the metric. In some embodiments, the HNN can leverage neighborhoods at multiple scales. This can, for example, allow for robustness and detail capture.

In some embodiments, the HNN can include hierarchical point set feature learning. The HNN can build hierarchal grouping of points and abstract larger and larger local regions along the hierarchy in some embodiments, for example. In some embodiments, the HNN can include a number of set abstraction levels. In some embodiments, a set of points is processed at each level and abstracted to produce a new set with fewer elements. In some embodiments, a set abstraction level can, in some embodiments, include three layers: sampling layer, grouping layer, and a local feature learner layer. In some embodiments, the local feature learner layer can be PointNet, for example. A set abstraction level can take an input of a N×(d+C) matrix that is from N points with d-dim coordinates and C-dim point feature and output a N×(d+C′) matrix of N′ subsampled points with d-dim coordinates and new C′-dim feature vectors that can summarize local context in some embodiments, for example.

The sampling layer can, in some embodiments, select or sample a set of points from input points. The HNN can define these selected/sampled points as centroids of local regions in some embodiments, for example. For example, for input points {x₁, x₂, . . . , xₙ} to the sampling layer, iterative farthest point sampling (FPS) can be used to choose a subset of points {xᵢ_{₁}, xᵢ_{₂}, . . . , xᵢ_{ₘ}} such that xᵢⱼ is the most distant point in metric distance from the set {xᵢ_{₁}, xᵢ_{₂}, . . . , xᵢ_{_{j−1}}} with respect to the rest points in some embodiments. This can advantageously provide, for example, better coverage of the whole point set given the same centroid number versus random sampling. This can also advantageously generate, for example, receptive fields in a way that is data dependent versus convolution neural networks (CNNs) which scan vector space independent of data distribution.

The grouping layer can determine one or more local region sets by determining neighboring points around each centroid in some embodiments, for example. In some embodiments, the input to this layer can be a point set of size N×(d+C) and coordinates of a centroids having the size N′×d. In some embodiments, the output of the grouping layer can include, for example groups of point sets having size N′×K×(d+C). Each group can correspond to a local region and K can be the number of points within a neighborhood of centroid points in some embodiments, for example. In some embodiments, K can vary from group to group. However, the next layer—the PointNet layer can convert the flexible number of points into a fixed length local region feature vector, for example. The neighborhood can, in some embodiments, be defined by metric distance, for example. Ball querying can determine all points within a radius to the query point in some embodiments, for example. An upper limit for K can be set. In an alternative embodiment, a K nearest neighbor (kNN) search can be used. kNN can determine a fixed number of neighboring points. However, ball query's local neighborhood can guarantee a fixed region scale, thus making one or more local region features more generalizable across space in some embodiments, for example. This can be preferable in some embodiments for semantic point labeling or other tasks that require local pattern recognition, for example.

In some embodiments, the local feature learner layer can encode local region patterns into feature vectors. For example, given that X=(M, d)) is a discrete metric space whose metric is inherited from a Euclidean space Xⁿ, where M⊆Rⁿ is the set of points and d is the distance metric, the local feature learner layer can determine functions ƒ that take X as input and output semantically interesting information regarding X. The function ƒ can be a classification function assigning a label to X or a segmentation function which assigns a per point label to each member of M.

Some embodiments can use PointNet as the local feature learner layer, which can, given an unordered point set {x₁, x₂, . . . , xₙ} with xᵢ ϵR^{d}, define a set function ƒ: X→R that maps a set of points to a vector such as, for example:

$$f ( {x}_{1} , {x}_{2} , … , {x}_{n} ) = γ ( {MAX}_{i = 1 , … , n} \{ h ( {x}_{i} ) \} ) .$$

In some embodiments, γ and h can be, multi-layer perceptron (MLP) networks for example, or other suitable alternatives known in the art. The function ƒ can be invariant to input point permutations and can approximate any continuous set function in some embodiments, for example. The response of h in some embodiments can be interpreted as a spatial encoding of a point. PointNet is described in “PointNet: Deep Learning on Point Sets for 3D Classification and Segmentation,” 2017 IEEE Conference on Computer Vision and Pattern Recognition (CVPR), 2017, pp. 77-85, by R. Q. Charles, H. Su, M. Kaichun and L. J. Guibas, the entirety of which is hereby incorporated by reference.

In some embodiments, the local feature learner layer can receive N′ local regions of points. The data size can be, for example, N′×K×(d+C). In some embodiments, each local region is abstracted by its centroid and local features that encodes the centroid's neighborhood, for example, in the output. The output data size can be, for example, N′×(d+C). Coordinates of points in a local region can be translated into a local frame relative to the centroid point in some embodiments: xᵢ^{(j)}=xᵢ^{(j)}−\hat{x}^{(j)} for i=1, 2, . . . , K and j=1, 2, . . . , d where \hat{x} is the centroid coordinate in some embodiments, for example. In some embodiments, using relative coordinates with point features can capture point-to-point relations in a local region, for example. In some embodiments, PointNet can be used for local pattern learning.

In some embodiments, the local feature learner can address non-uniform density in the input point set through density adaptive layers, for example. Density adaptive layers can learn to combine features of differently scaled regions when the input sampling density changes. In some embodiments, the density adaptive hierarchical network is a PointNet++ network, for example. Density adaptive layers can include multi-scale grouping (“MSG”) or Multi-resolution grouping (“MRG”) in some embodiments, for example.

In MSG, multiscale patterns can be captured by applying grouping layers with different scales followed by extracting features of each scale in some embodiments. Extracting features of each scale can be performed by utilizing PointNet in some embodiments, for example. In some embodiments, features at different scales can be concatenated to provide a multi-scale feature, for example. In some embodiments, the HNN can learn optimized multi-scale feature combining by training. For example, random input dropout in which random input points are dropped input points with randomized probability can be used. As an example, a dropout ratio of Θ that is uniformly sampled from [0,p], where p is less than or equal to 1 can be used in some embodiments, for example. As an example, p can be set to 0.95 in some cases so that empty point sets are not generated. Other suitable values can be used in some embodiments, for example.

In MRG, features of one region at a level Lᵢ, for example, can be a concatenation of two vectors, with a first vector obtained by summarizing features at each subregion from a lower level Lᵢ₋₁ in some embodiments, for example. This can be accomplished using the set abstraction level. A second vector can be the feature obtained by directly processing local region raw points using, for example a single PointNet in some embodiments. In cases where a local region density is low, the second vector can be weighted more in some embodiments since the first vector contains fewer points and includes sampling deficiencies. In cases where a local region density is high, for example, the first vector can be weighted more in some embodiments since the first vector can provide finer details due to inspection at higher resolutions recursively at lower levels.

In some embodiments, point features can be propagated for set segmentation. For example, in some embodiments a hierarchical propagation strategy can be used. In some embodiments, feature propagation can include propagating point features from Nₗ×(d+C) points to Nₗ₋₁ points, where Nₗ₋₁ and Nₗ (Nₗ is less than or equal to Nₗ₋₁) are point set size of input and output of set abstraction level 1. In some embodiments, feature propagation can be achieved through interpolation of feature values ƒ of Nₗ points at coordinates of the Nₗ₋₁ points. In some embodiments, an inverse distance weighted average based on k nearest neighbors can be used, for example (p=2, k=3 in equation below; other suitable values can be used). Interpolated features on Nₗ₋₁ can be concatenated with skip linked point features from the set abstraction level in some embodiments, for example. In some embodiments, concatenated features can be passed through a unit PointNet, which can be similar to a one-by-one convolution in convolutional neural networks, for example. Shared fully connected and ReLU layers can be applied to update each point's feature vector in some embodiments, for example. In some embodiments, the process can be repeated until propagated features to the original set of points are determined.

$${f}^{( j )} ( x ) = \frac{{\sum }_{i = 1}^{k} {w}_{i} ( x ) {f}_{i}^{( j )}}{{\sum }_{i = 1}^{k} {w}_{i} ( x )} where {w}_{i} ( x ) = \frac{1}{{d ( x , {x}_{i} )}^{p}} , j = 1 , … , C$$

In some embodiments, the computer-implemented method can implement one or more neural networks as disclosed or as are known in the art. Any specific structures and values with respect to one or more neural networks and any other features as disclosed herein are provided as examples only, and any suitable variants or equivalents can be used. In some embodiments, one or more neural network models can be implemented on the base of the Pytorch-geometric package as an example.

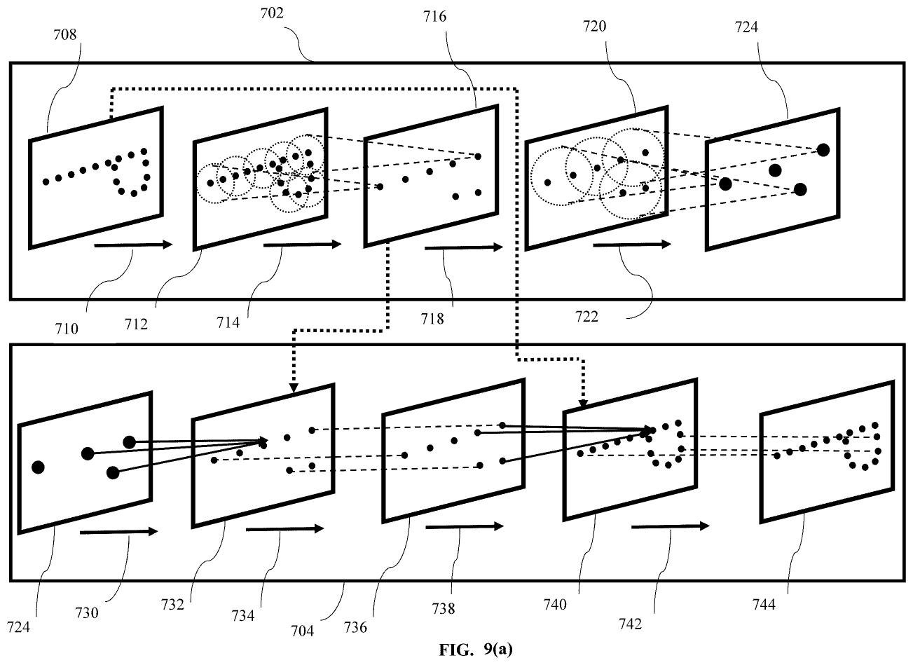

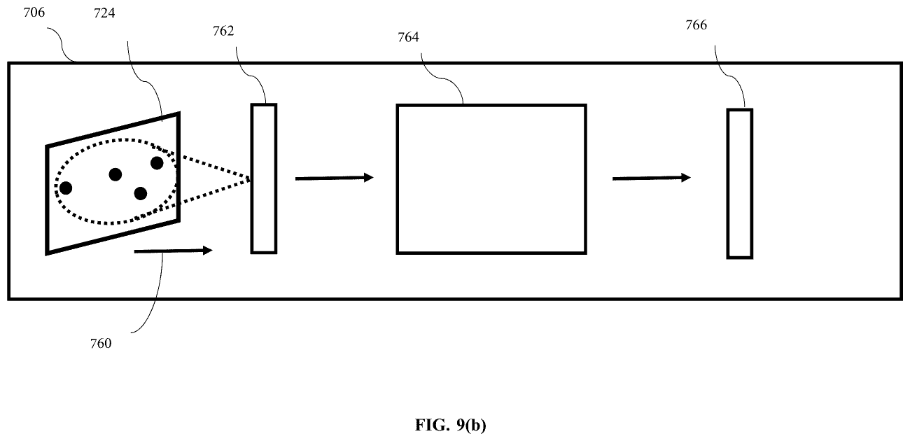

FIG. 9(a) and FIG. 9(b) illustrate an example of HNN in some embodiments. The HNN can include a hierarchal point set feature learner 702, the output of which can be used to perform segmentation 704 and/or classification 706. The hierarchal point set feature learner 702 example uses points in 2D Euclidean space as an example, but can operate on input 3D images in three dimensions. As illustrated in the example of FIG. 9(a), the HNN can receive an input image 708 with (N, d+C) and perform a first sampling and grouping operation 710 to generate a first sampled and grouped image 712 with (N₁, K, d+C), for example. The HNN can then provide the first sampled and grouped image 712 to PointNet at 714 to provide first abstracted image 716 with (N₁, d+C₁). The first abstracted image 716 can undergo sampling and grouping 718 to provide a second sampled and grouped image 720 with (N₂, K, d+C₁). The second sample and grouped image 720 can be provided to a PointNet neural network 722 to output a second abstracted image 724 with (N₂, d+C2).

In some embodiments, the second abstracted image 724 can be segmented by HNN segmentation 704. In some embodiments, the HNN segmentation 704 can take the second abstracted image 724 and perform a first interpolation 730, the output of which can be concatenated with the first abstracted image 716 to provide a first interpolated image 732 with (N₁, d+C₂+C₁). The first interpolated image 732 can be provided to a unit PointNet at 734 to provide first segment image 736 with (N₁, d+C₃). The first segment image 736 can be interpolated at 738, the output of which can be concatenated with the input image 708 to provide a second interpolated image 740 with (N₁, d+C₃+C). The second interpolated image 740 can be provided to a unit PointNet 742 to provide a segmented image 744 with (N, k). The segmented image 744 can provide per-point scores, for example.

As illustrated in the example of FIG. 9(b), the second abstracted image 724 can be classified by HNN classification 706 in some embodiments. In some embodiments, HNN classification can take second abstracted image 724 and provide it to a PointNet network 760, the output 762 of which can be provided to one or more fully connected layers such as connected layers 764, the output of which can provide class scores 766.

Some embodiments of the computer-implemented method can include determining, using a displacement value trained neural network, a margin line proposal from a base margin line and the inner representation of the 3D digital model.

In some embodiments, the base margin line can be precomputed once per network type. In some embodiments, the network types can include molar and premolar. Other suitable network types can be used in some embodiments for example. In some embodiments, the network types can include other types. In some embodiments, the same base margin line can be used as an initial margin line for each scan. In some embodiments, the base margin line is 3 dimensional. In some embodiments, the base margin line can be determined based on margin lines from a training dataset used to train the inner representation trained neural network and the displacement value trained neural networks. In some embodiments, the base margin line can be a precomputed mean or average of the training dataset margin lines. In some embodiments any type of mean or average can be used.

In some embodiments, the margin line proposal can be a free-form margin line proposal. In some embodiments, a displacement value trained neural network can include a decoder neural network. In some embodiments, the decoder neural network can concatenate the inner representation with specific point coordinates to implement guided decoding. In some embodiments, the guided decoding can generate a closed surface as described in “A Papier-Mache Approach to Learning 3D Surface Generation,” by T. Groueix, M. Fisher, V. G. Kim, B. C. Russell and M. Aubry, 2018 IEEE/CVF Conference on Computer Vision and Pattern Recognition, 2018, pp. 216-224, the entirety of which is hereby incorporated by reference.

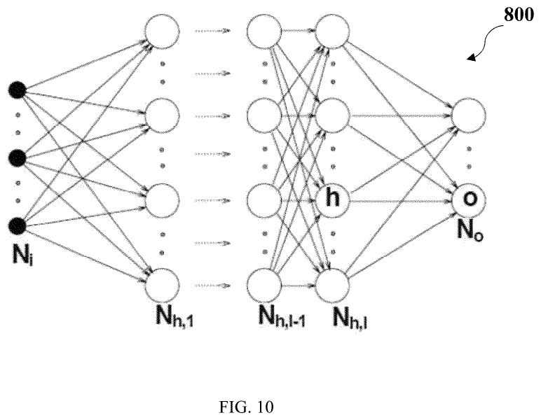

In some embodiments, the decoder neural network can include a deep neural network (“DNN”). Referring now to FIG. 10, which is a high-level block diagram showing structure of a deep neural network (DNN) 800 according to some embodiments of the disclosure. DNN 800 includes multiple layers Nᵢ, N_{h,l}, N_{h,l-1}, N_{h,l}, Nₒ, etc. The first layer Nᵢ is an input layer where one or more dentition scan data sets can be ingested. The last layer Nₒ is an output layer. The deep neural networks used in the present disclosure may output probabilities and/or full 3D margin line proposal. For example, the output can be a probability vector that includes one or more probability values of each feature or aspect of the dental models belonging to certain categories. Additionally, the output can be a margin line proposal.

Each layer N can include a plurality of nodes that connect to each node in the next layer N+1. For example, each computational node in the layer N_{h, l-1} connects to each computational node in the layer N_{h,l}. The layers N_{h,l}, N_{h,l-1}, N_{h,l}, between the input layer Nᵢ and the output layer Nₒ are hidden layers. The nodes in the hidden layers, denoted as “h” in FIG. 10, can be hidden variables. In some embodiments, DNN 800 can include multiple hidden layers, e.g., 24, 30, 50, etc.

In some embodiments, DNN 800 may be a deep feedforward network. DNN 800 can also be a convolutional neural network, which is a network that uses convolution in place of the general matrix multiplication in at least one of the hidden layers of the deep neural network. DNN 800 may also be a generative neural network or a generative adversarial network. In some embodiments, training may use training data set with labels to supervise the learning process of the deep neural network. The labels are used to map a feature to a probability value of a probability vector. Alternatively, training may use unstructured and unlabeled training data sets to train, in an unsupervised manner, generative deep neural networks that do not necessarily require labeled training data sets.

In some embodiments, the DNN can be a multi-layer perceptron (“MLP”). In some embodiments, the MLP can include 4 layers. In some embodiments, the MLP can include a fully connected MLP. In some embodiments, the MLP utilizes BatchNorm normalization.

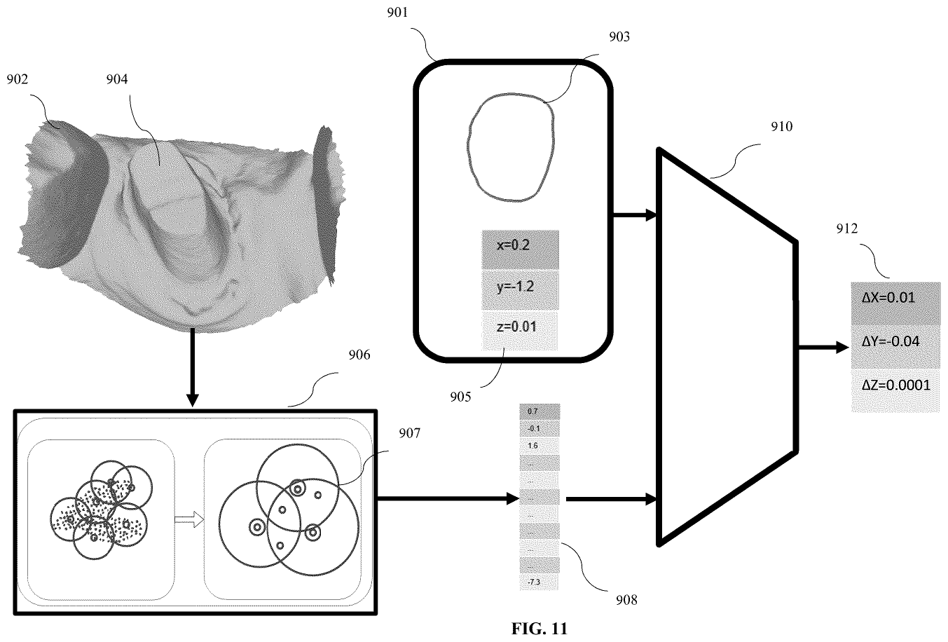

FIG. 11 shows a diagram of a computer-implemented method of automatic margin line proposal in some embodiments as an example. In some embodiments, prior to commencing margin line proposal for any 3D digital model, the computer-implemented method can precompute 901 a base margin line 903 in three dimensions, with each point of the base margin line 903 having 3D coordinates such as coordinates 905, for example. The computer-implemented method can receive a 3D digital model 902 of at least a portion of a jaw. The 3D digital model can, in some embodiments, be in the form of a 3D point cloud. The 3D digital model can include a preparation tooth 904, for example. The computer-implemented method can use an inner representation trained neural network 906 to determine an inner representation 908 of the 3D digital model. In some embodiments, the inner representation trained neural network 906 can be a neural network that performs grouping and sampling 907 and other operations on a 3D digital model, such as an HNN, in some embodiments, for example. In some embodiments, the computer-implemented method can, using a displacement value trained neural network 910, determine a margin line proposal from the base margin line 903 and the inner representation 908 of the 3D digital model. In some embodiments, the displacement value trained neural network can provide, for example, one or more three dimensional displacement values 912 for digital surface points of the base margin line 903.

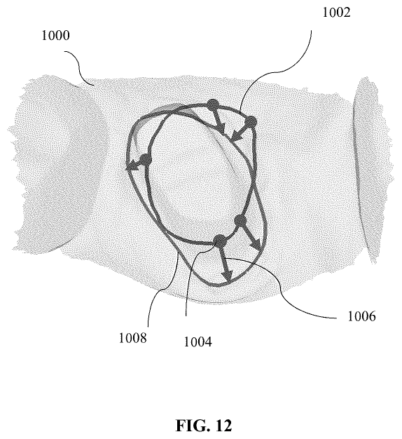

In some embodiments, the displacement value trained neural network can determine a margin line displacement value in three dimensions from the base margin line. In some embodiments, the displacement value trained neural network uses a BilateralChamferDistance as a loss function. In some embodiments, the computer-implemented method can move one or points of the base margin line by a displacement value to provide the margin line proposal. FIG. 12 shows an illustration of an example in some embodiments of adjusting the base margin line 1002 of a 3D digital model 1000. In the example, one or more base margin line points such as base margin line point 1004 can be displaced by a displacement value and direction 1006. Other base margin line points can be similarly adjusted by their corresponding displacement values and directions to form the margin line proposal 1008, for example.

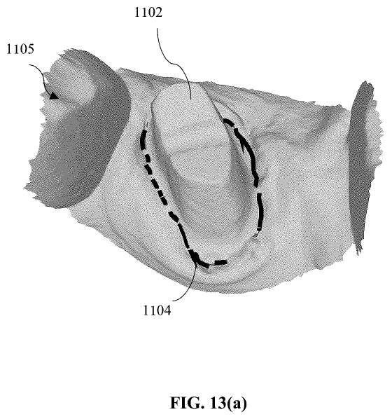

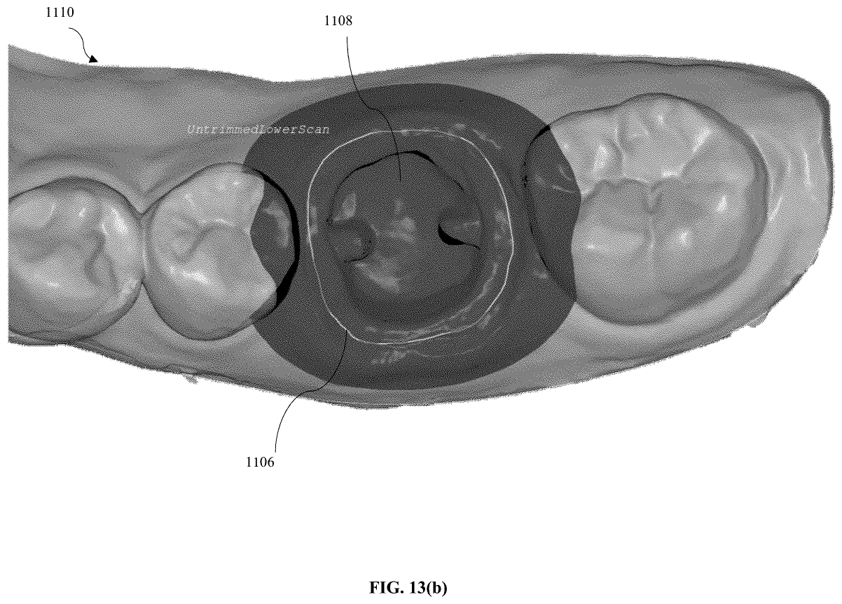

FIG. 13(a) illustrates one example of a proposed digital margin line 1104 for a digital preparation tooth 1102 of 3D digital model 1105. As can be seen in the figure, the margin line proposal can be made even in cases where the margin line is partially or completely covered by gum, blood, saliva, or other elements. FIG. 13(b) illustrates another example of a proposed digital margin line 1106 for digital preparation tooth 1108 of 3D digital dental model 1110. In some embodiments, the proposed margin line is displayed on the 3D digital model and can be manipulated by a user such as a dental technician or doctor using an input device to make adjustments to the margin line proposal.

In some embodiments, the inner representation trained neural network and the displacement value trained neural network can be trained using the same training dataset. In some embodiments, the training dataset can include one or more training samples. In some embodiments, the training dataset can include 70,000 training samples. In some embodiments, the one or more training samples each can include an occlusal direction, preparation die center, and buccal direction as a normalized positioning and orientation for each sample. In some embodiments, the occlusal direction, preparation die center, and buccal direction can be set manually. In some embodiments, the training dataset can include an untrimmed digital surface of the jaw and a target margin line on a surface of the corresponding trimmed digital surface. In some embodiments, the target margin line can be prepared by a technician. In some embodiments, training can use regression. In some embodiments, training can include using a loss-function to compare the margin line proposal with the target margin line. In some embodiments, the loss function can be a Chamfer-loss function. In some embodiments, the Chamfer-loss function can include:

$${d}_{CD} ( {S}_{1} , {S}_{2} ) = {\sum }_{x ∈ {S}_{1}} {min}_{y ∈ {S}_{2}} {‖ x - y ‖}_{2}^{2} + {\sum }_{x ∈ {S}_{2}} {min}_{y ∈ {S}_{1}} {‖ x - y ‖}_{2}^{2}$$

In some embodiments, training can be performed on a computing system can include at least one graphics processing unit (“GPU”). In some embodiments, the GPU can include two 2080-Ti Nvidia GPUs, for example. Other suitable GPU types, numbers, and equivalents can be used.

In some embodiments, the computer-implemented method can be performed automatically. Some embodiments can further include displaying the free-form margin line on the 3D digital model. In some embodiments, the free-form margin line can be adjusted by a user using an input device.

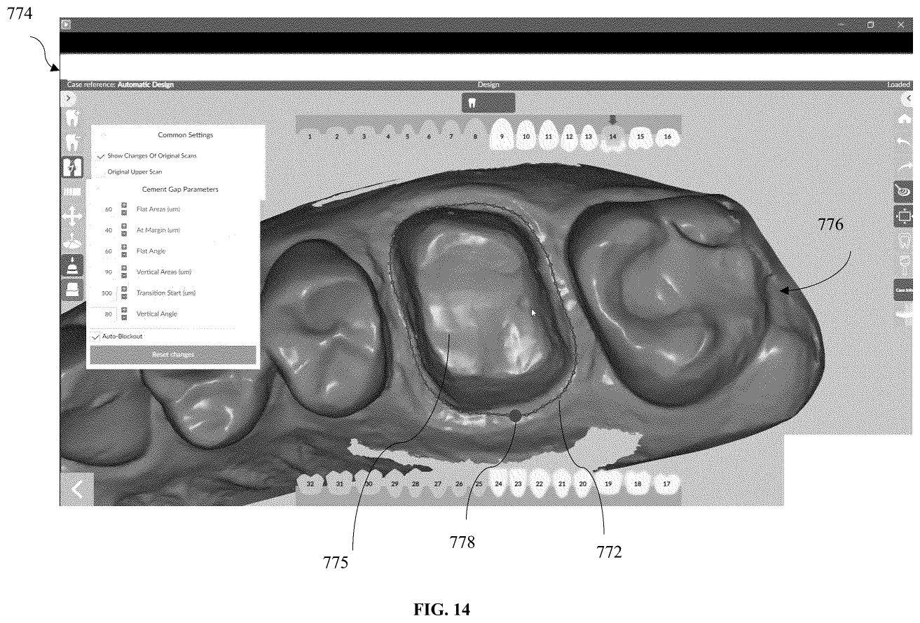

In some embodiments, the adjustable margin line can be displayed to the dentist or other user for an initial adjustment. In some embodiments, adjustment can include moving one or more portions of the margin line based on the dentist or other user's input. In some embodiments, adjustment can include discarding the adjustable margin line and allowing the dentist to manually provide the margin line. In some embodiments, the computer-implemented method can display at least a portion of the 3D virtual dental model of a patient's dentition and the automatically determined margin line proposal and allow a dentist or other user to modify the determined margin line. FIG. 14 illustrates an example in which a determined margin line proposal 772 in GUI 774 for the virtual preparation tooth 775 in the 3D virtual dental model 776. In some embodiments, determining an adjustable margin line occurs prior to generating the 3D virtual dental restoration. Accordingly, during the initial adjustment for the margin line, no 3D virtual dental restoration appears, so that the proposed (determined) margin line is visible. In some embodiments, GUI 774 can provide virtual handles 778 to allow a user to adjust the automatically determined margin line. This can advantageously allow, for example, correction of the automatically determined margin line. In some embodiments, automatically determining the margin line can be performed while the patient is in the dental office.

In some embodiments, the automated virtual restoration design can include determining an insertion direction of a restoration onto the preparation tooth based on the adjustable margin line. Examples of automatically determining an insertion direction based on the adjustable margin line can be found in U.S. application Ser. No. 16/918,586 of Leeson et al., the entirety of which is hereby incorporated by reference. In some embodiments, determining the insertion direction can be performed while the patient is in the dental office.

In some embodiments, the automated virtual restoration design can include determining an inner surface of the virtual restoration based on a cement space. One or more examples of automatically determining an inner surface of the virtual restoration to account for cement space. on the 3D virtual dental model can be found in U.S. application Ser. No. 16/918,586 of Leeson et al., the entirety of which is hereby incorporated by reference. In some embodiments, determining the inner surface of the virtual restoration based on the cement space can be performed while the patient is in the dental office. In some embodiments, cement space can include space between the physical preparation tooth and the physical restoration. In some embodiments, cement space can include determining an inner surface of the virtual restoration. In some embodiments, the inner surface can include an offset from an outer surface of the virtual restoration, the offset comprising the cement gap. In some embodiments, accounting for cement space can include providing space for cement around the virtual margin and along one or more horizontal and vertical surfaces of the virtual restoration. In some embodiments, the automated virtual restoration design can include a tool radius compensation for a milling tool. In some embodiments, determining an inner surface of the virtual restoration based on a cement space can be performed while a patient is in the dental office.

In some embodiments, the automated virtual restoration design can include automatically generating a 3D virtual restoration based on the adjustable margin line. Examples of automatically generating a 3D virtual restoration can be found in U.S. Pat. No. 11,291,532B2 to Azernikov et al. and U.S. Pat. No. 11,007,040B2 to Azernikov et al., the entireties of each of which are hereby incorporated by reference. In some embodiments, automatically generating the 3D virtual restoration can be performed while the patient is in the dental office. In some embodiments, the 3D virtual restoration can include a virtual crown. In some embodiments, the 3D virtual restoration can include a virtual bridges.

Some embodiments can include generating, using a trained deep neural network, a virtual 3D dental prosthesis model based on the virtual 3D dental model. Some embodiments can include automatically generating a 3D digital dental prosthesis model (the virtual 3D dental prosthesis model) in the 3D digital dental model using a trained generative deep neural network. One example of generating a dental prosthesis using a deep neural network is described in U.S. patent application Ser. No. 15/925,078, now U.S. Pat. No. 11,007,040, the entirety of which is hereby incorporated by reference. Another example of generating a dental prosthesis using a deep neural network is described in U.S. patent application Ser. No. 15/660,073, the entirety of which is hereby incorporated by reference.

Example embodiments of methods and computer-implemented systems for generating a 3D model of a dental prosthesis using deep neural networks are described herein. Certain embodiments of the methods can include: training, by one or more computing devices, a deep neural network to generate a first 3D dental prosthesis model using a training data set; receiving, by the one or more computing devices, a patient scan data representing at least a portion of a patient's dentition; and generating, using the trained deep neural network, the first 3D dental prosthesis model based on the received patient scan data.

The training data set can include a dentition scan data set with preparation site data and a dental prosthesis data set. A preparation site on the gum line can be defined by a preparation margin or margin line on the gum. The dental prosthesis data set can include scanned prosthesis data associated with each preparation site in the dentition scan data set.

The scanned prosthesis can be scans of real patients' crowns created based on a library tooth template, which can have 32 or more tooth templates. The dentition scan data set with preparation site data can include scanned data of real preparation sites from patients' scanned dentition.

In some embodiments, the training data set can include a natural dentition scan data set with digitally fabricated preparation site data and a natural dental prosthesis data set, which can include segmented tooth data associated with each digitally fabricated preparation site in the dentition scan data set. The natural dentition scan data set can have two main components. The first component is a data set that includes scanned dentition data of patients' natural teeth. Data in the first component includes all of the patients' teeth in its natural and unmodified digital state. The second component of the natural dentition scan data is a missing-tooth data set with one or more teeth removed from the scanned data. In place of the missing-tooth, a deep neural network fabricated preparation site can be placed at the site of the removed tooth. This process generates two sets of dentition data: a full and unmodified dentition scan data of patients' natural teeth; and a missing-tooth data set (natural dental prosthesis data set) in which one or more teeth are digitally removed from the dentition scan data.

In some embodiments, the method further includes generating a full arch digital model and segmenting each tooth in the full arch to generate natural crown data for use as training data. The method can also include: training another deep neural network to generate a second 3D dental prosthesis model using a natural dentition scan data set with digitally fabricated preparation site data and a natural dental prosthesis data set; generating, using the other deep neural network, the second 3D dental prosthesis model based on the received patient scan data; and blending together features of the first and second 3D dental prosthesis models to generated a blended 3D dental prosthesis model.

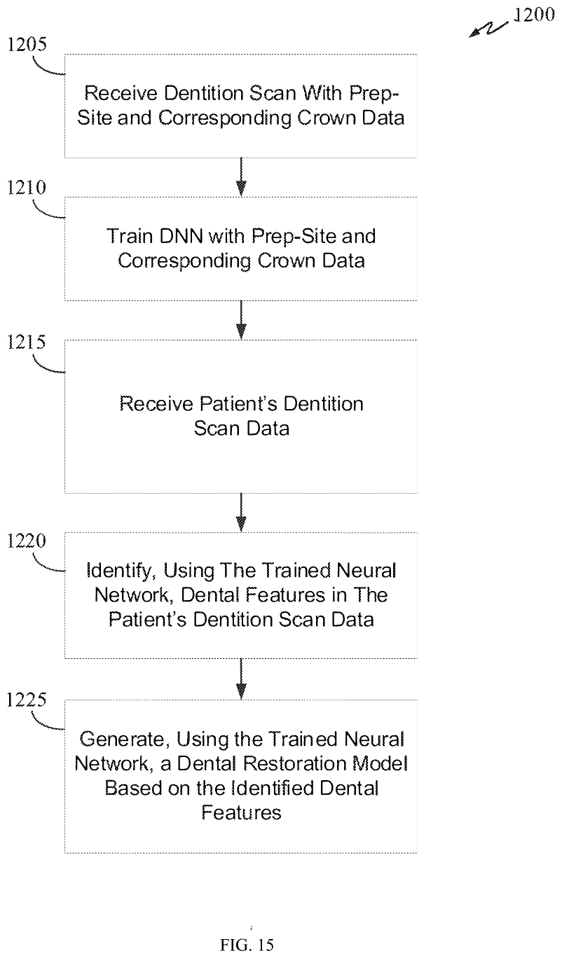

FIG. 15 illustrates a dental prosthesis generation process 1200 using a deep neural network (DNN). Process 1200 starts at 1205 where a dentition scan data set is received or ingested into a database. The dentition scan data set can include one or more scan data sets of real patient's dentitions with dental preparation sites and technician-generated (non-DNN generated) dental prostheses created for those preparation sites. A dental preparation site (also referred to as a tooth preparation or a prepared tooth) is a tooth, a plurality of teeth, or an area on a tooth that has been prepared to receive a dental prosthesis (e.g., crown, bridge, inlay, etc.). A technician or a non-DNN generated dental prosthesis is a dental prosthesis mainly designed by a technician. Additionally, a technician-generated dental prosthesis can be designed based on a dental template library having a plurality of dental restoration templates. Each tooth in an adult mouth can have one or more dental restoration templates in the dental template library.

In some embodiments, the received dentition scan data set with dental preparation sites can include scan data of real patients' dentition having one or more dental preparation sites. A preparation site can be defined by a preparation margin. The received dentition scan data set can also include scan data of dental prostheses once they are installed on their corresponding dental preparation sites. This data set can be referred to as a dental prosthesis data set. In some embodiments, the dental prosthesis data set can include scan data of technician-generated prostheses before they are installed.

In some embodiments, each dentition scan data set received may optionally be preprocessed before using the data set as input of the deep neural network. Dentition scan data are typically 3D digital image or file representing one or more portions of a patient's dentition. The 3D digital image (3D scan data) of a patient's dentition can be acquired by intraorally scanning the patient's mouth. Alternatively, a scan of an impression or of a physical model of the patient's teeth can be made to generate the 3D scan data of a patient's dentition. In some embodiments, the 3D scan data can be transformed into a 2D data format using, for example, 2D depth maps and/or snapshots.

At 1210, a deep neural network can be trained (by the computer-implemented method or another process for example) using a dentition scan data set having scan data of real dental preparation sites and their corresponding technician-generated dental prostheses-post installation and/or before installation. The above combination of data sets of real dental preparation sites and their corresponding technician-generated dental prostheses can be referred to herein as a technician-generated dentition scan data set. In some embodiments, the deep neural network can be trained using only technician-generated dentition scan data set. In other words, the training data only contain technician-generated dental prostheses, which were created based on one or more dental restoration library templates.

A dental template of the dental restoration library can be considered to be an optimum restoration model as it was designed with specific features for a specific tooth (e.g., tooth #3). In general, there are 32 teeth in a typical adult's mouth. Accordingly, the dental restoration library can have at least 32 templates. In some embodiments, each tooth template can have one or more specific features (e.g., sidewall size and shape, buccal and lingual cusp, occlusal surface, and buccal and lingual arc, etc.) that may be specific to one of the 32 teeth. For example, each tooth in the restoration library is designed to include features, landmarks, and directions that would best fit with neighboring teeth, surrounding gingiva, and the tooth location and position within the dental arch form. In this way, the deep neural network can be trained to recognize certain features (e.g., sidewall size and shape, cusps, grooves, pits, etc.) and their relationships (e.g., distance between cusps) that may be prominent for a certain tooth.

In some embodiments, the computer-implemented method or any other process may train the deep neural network to recognize one or more dentition categories are present or identified in the training data set based on the output probability vector. For example, assume that the training data set contains a large number of depth maps representing patients' upper jaws and/or depth maps representing patients' lower jaws. The computer-implemented method or another process can use the training data set to train the deep neural network to recognize each individual tooth in the dental arch form. Similarly, the deep neural network can be trained to map the depth maps of lower jaws to a probability vector including probabilities of the depth maps belonging to upper jaw and lower jaw, where the probability of the depth maps belonging to lower jaw is the highest in the vector, or substantially higher than the probability of the depth maps belonging to upper jaw.

In some embodiments, the computer-implemented method or another process can train a deep neural network, using dentition scan data set having one or more scan data sets of real dental preparation sites and corresponding technician-generated dental prostheses, to generate full 3D dental restoration model. In this way, the DNN generated 3D dental restoration model inherently incorporates one or more features of one or more tooth templates of the dental restoration library, which may be part of database 150.

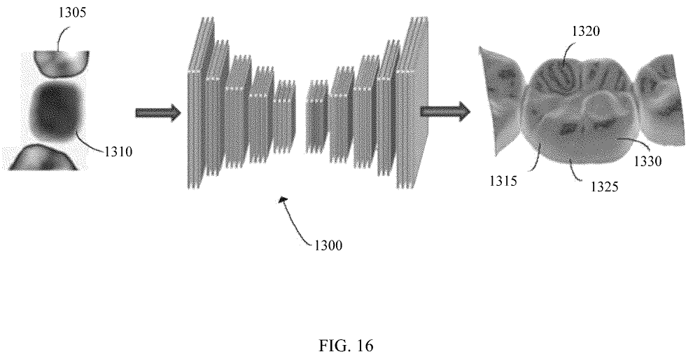

The computer-implemented method or another process can train a deep neural network such as the one discussed in FIG. 10, FIG. 16, FIG. 14(a), or other neural network to generate a 3D model of dental restoration using only the technician-designed dentition scan data set. In this way, the DNN generated 3D dental prosthesis will inherently include one or more features of dental prosthesis designed by a human technician using the library template. In some embodiments, the computer-implemented method or another process can train the deep neural network to output a probability vector that includes a probability of an occlusal surface of a technician-generated dental prosthesis representing the occlusal surface of a missing tooth at the preparation site or margin. Additionally, the computer-implemented method or another process can train a deep neural network to generate a complete 3D dental restoration model by mapping the occlusal surface having the highest probability and margin line data from the scanned dentition data to a preparation site. Additionally, the computer-implemented method or another process can train the deep neural network to generate the sidewall of the 3D dental restoration model by mapping sidewalls data of technician-generated dental prostheses to a probability vector that includes a probability of that one of the sidewalls matches with the occlusal surface and the margin line data from the preparation site.

Referring again to FIG. 15, to generate a new 3D model of a dental prosthesis for a new patient, the new patient's dentition scan data (e.g., scanned dental impression, physical model, or intraoral scan) received and ingested at 1215. In some embodiments, the new patient's dentition scan data can be preprocessed to transform 3D image data into 2D image data, which can make the dentition scan data easier to ingest by certain neural network algorithms. At 1220, using the previously trained deep neural network, one or more dental features in the new patient's dentition scan data are identified. The identified features can be a preparation site, the corresponding margin line, adjacent teeth and corresponding features, and surrounding gingiva for example.

At 1225, using the trained deep neural network, a full 3D dental restoration model can be generated based on the identified features at 1220. In some embodiments, the trained deep neural network can be tasked to generate the full 3D dental restoration model by: generating an occlusal portion of a dental prosthesis for the preparation site; obtaining the margin line data from the generated margin proposal as described previously or from patient's dentition scan data; optionally optimizing the margin line; and generating a sidewall between the generated occlusal portion and the margin line. Generating an occlusal portion can include generating an occlusal surface having one or more of a mesiobuccal cusp, buccal grove, distobuccal cusp, distal cusp, distobuccal groove, distal pit, lingual groove, mesiolingual cusp, etc.

In some embodiments, the trained deep neural network can obtain the margin line data from the generated margin proposal as described previously or from the patient's dentition scan data. In some embodiments, the trained deep neural network can optionally modify the contour of the obtained margin line by comparing and mapping it with thousands of other similar margin lines (e.g., margin lines of the same tooth preparation site) having similar adjacent teeth, surrounding gingiva, etc.

To generate the full 3D model, the trained deep neural network can generate a sidewall to fit between the generated occlusal surface and the margin line. This can be done by mapping thousands of sidewalls of technician-generated dental prostheses to the generated occlusal portion and the margin line. In some embodiments, a sidewall having the highest probability value (in the probability vector) can be selected as a base model in which the final sidewall between occlusal surface and the margin line will be generated.

FIG. 16 illustrates example input and output of the trained deep neural network 1300 (e.g., GAN) in accordance with some embodiments of the present disclosure. As shown, an input data set 1305 can the new patient's dentition scan having a preparation site 1310. Using the trained one or more deep neural network 1300, dental restoration server can generate a (DNN-generated) 3D model of a dental restoration 1315. DNN-generated dental prosthesis 1315 includes an occlusal portion 1320, a margin line portion 1325, and a sidewall portion 1330. In some embodiments, the deep neural network can generate the sidewall for prosthesis 1315 by analyzing thousands of technician-generated dental prostheses—which were generated based on one or more library templates—and mapping them to preparation site 1310. Finally, the sidewall having the highest probability value can be selected as a model to generate sidewall 1330.

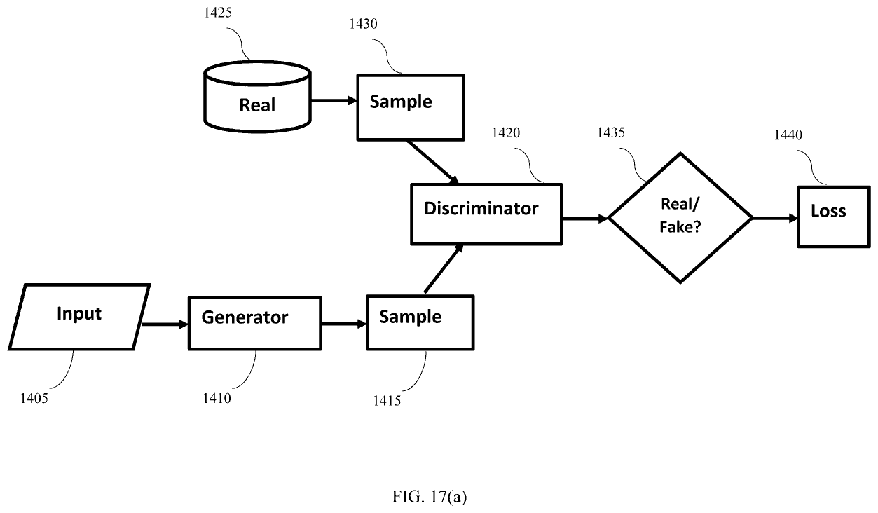

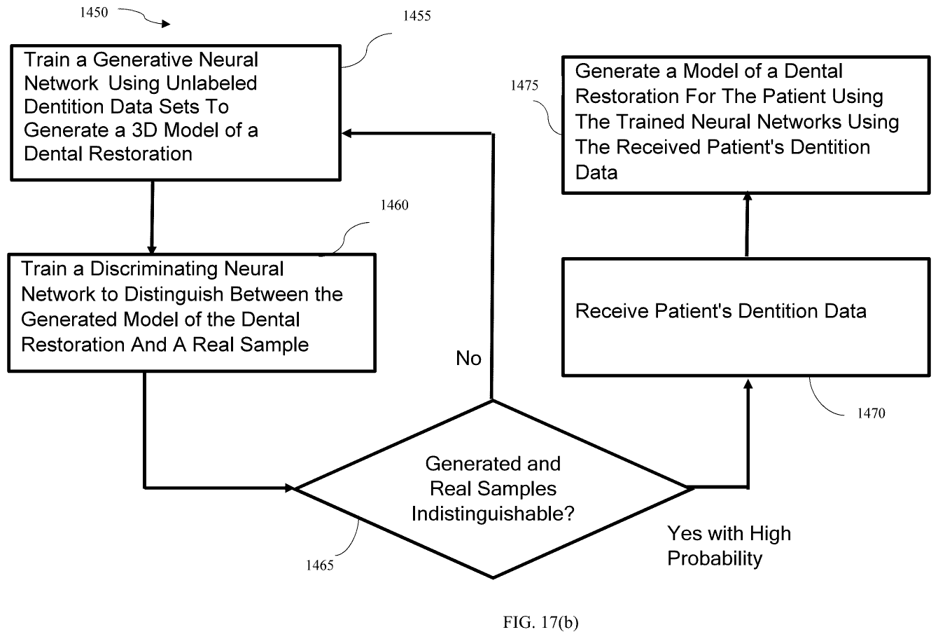

FIG. 17(a) is a high-level block diagram illustrating a structure of a generative adversarial network (GAN network) that can be employed to identify and model dental anatomical features and restorations, in accordance with some embodiments of the present disclosure. On a high level, GAN network uses two independent neural networks against each other to generate an output model that is substantially indistinguishable when compared with a real model. In other words, GAN network employs a minimax optimization problem to obtain convergence between the two competing neural networks. GAN network includes a generator neural network 1410 and a discriminator neural network 1420. In some embodiments, both neural network 1410 and discriminator neural network 1420 are deep neural networks structured to perform unstructured and unsupervised learning. In the GAN network, both the generator network 1410 and the discriminator network (discriminating deep neural network) 1420 are trained simultaneously. Generator network 1410 is trained to generate a sample 1415 from the data input 1405. Discriminator network 1420 is trained to provide a probability that sample 1415 belongs to a training data sample 1430 (which comes from a real sample, real data 1425) rather than one of the data sample of input 1405. Generator network 1410 is recursively trained to maximize the probability that discriminator network 1420 fails to distinguish apart (at 1435) a training data set and an output sample generated by generator 1410.

At each iteration, discriminator network 1420 can output a loss function 1440, which is used to quantify whether the generated sample 1415 is a real natural image or one that is generated by generator 1410. Loss function 1440 can be used to provide the feedback required for generator 1410 to improve each succeeding sample produced in subsequent cycles. In some embodiments, in response to the loss function, generator 1410 can change one or more of the weights and/or bias variables and generate another output

In some embodiments, the computer-implemented method or another process can simultaneously train two adversarial networks, generator 1410 and discriminator 1420. The computer-implemented method or another process can train generator 1410 using one or more of a patient's dentition scan data sets to generate a sample model of one or more dental features and/or restorations. For example, the patient's dentition scan data can be 3D scan data of a lower jaw including a prepared tooth/site and its neighboring teeth. Simultaneously, the computer-implemented method or another process can train discriminator 1420 to distinguish a generated a 3D model of a crown for the prepared tooth (generated by generator 1410) against a sample of a crown from a real data set (a collection of multiple scan data set having crown images). In some embodiments, GAN networks are designed for unsupervised learning, thus input 1405 and real data 1425 (e.g., the dentition training data sets) can be unlabeled.

FIG. 17(b) is a flow chart of a method 1450 for generating a 3D model of a dental restoration in accordance with some embodiments of the present disclosure. Method 1450 can be performed by the computer-implemented method or another process on the dental restoration server or one or more other computers in the cloud computing environment. The instructions, processes, and algorithms of method 1450 may be stored in memory of a computing device, and when executed by a processor, they enable computing device to perform the training of one or more deep neural networks for generating 3D dental prostheses. Some or all of the processes and procedures described in method 1450 may be performed by one or more other entities or processes within dental restoration server or within another remote computing device. In addition, one or more blocks (processes) of method 1450 may be performed in parallel, in a different order, or even omitted.

At 1455, the computer-implemented method or another process may train a generative deep neural network (e.g., GAN generator 1410) using unlabeled dentition data sets to generate a 3D model of a dental prosthesis such as a crown. In some embodiments, labeled and categorized dentition data sets may be used, but not necessary. The generative deep neural network may be implemented by the computer-implemented method or another process or in a separate and independent neural network, within or outside of the dental restoration server.

At 1460, and at substantially the same, the computer-implemented method or another process may also train a discriminating deep neural network (e.g., discriminator 1420) to recognize that the dental restoration generated by the generative deep neural network is a model versus a digital model of a real dental restoration. In the recognition process, the discriminating deep neural network can generate a loss function based on comparison of a real dental restoration and the generated model of the dental restoration. The loss function provides a feedback mechanism for the generative deep neural network. Using information from the outputted loss function, the generative deep neural network may generate a better model that can better trick the discriminating neural network to think the generated model is a real model.

The generative deep neural network and the discriminating neural network can be considered to be adverse to each other. In other words, the goal of the generative deep neural network is to generate a model that cannot be distinguished by the discriminating deep neural network to be a model belonging a real sample distribution or a fake sample distribution (a generated model). At 1465, if the generated model has a probability value indicating that it is most likely a fake, the training of both deep neural networks repeats and continues again at 1455 and 1460. This process continues and repeats until the discriminating deep neural network cannot distinguish between the generated model and a real model. In other words, the probability that the generated model is a fake is very low or that the probability that the generated model belong to a distribution of real samples is very high.

Once the deep neural networks are trained, method 1400 is ready to generate a model of a dental restoration based on the patient's dentition data set, which is received at 1470. At 1475, a model of the patient's dentition data set is generated using the received patient's dentition data set.

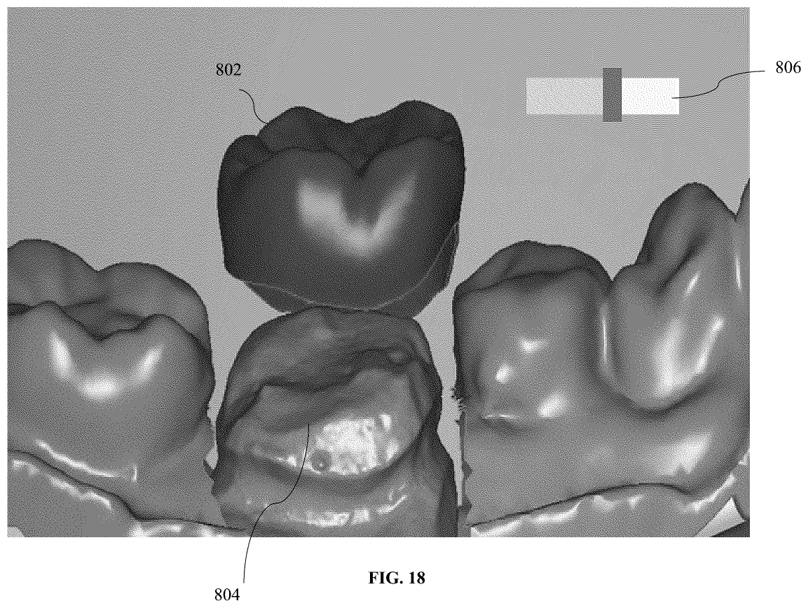

In some embodiments, the automated virtual restoration design can include determining an insertion direction of the generated 3D virtual restoration. Examples of automatically determining an insertion direction of the generated 3D virtual restoration can be found in U.S. Pat. No. US20210304874A1 to Nikolskiy et al., the entirety of which is hereby incorporated by reference. In some embodiments, the automated virtual restoration design can illustrate pulling restoration to the margin along the determined insertion path. FIG. 18 illustrates an example in some embodiments of a virtual crown 802 lifted along an insertion path of a virtual preparation tooth 804. In some embodiments, the lifted crown can be moved along the insertion path using a GUI element such as a slider 806 or other equivalent GUI element.

Some embodiments of the computer-implemented method of virtual dental restoration design automation can include detecting the presence or absence of physical preparation tooth issues during the automated virtual restoration design. In some embodiments, the virtual dental restoration design automation detecting the presence or absence of physical preparation tooth issues during the automated virtual restoration design can be performed while the patient is in the dental office.

In some embodiments the one or more physical preparation tooth issues can include automatically determining one or more undercut regions of the virtual preparation tooth. One or more examples of automatically determining one or more undercut regions of the virtual preparation tooth can be found in U.S. application Ser. No. 16/918,586, previously incorporated by reference. As described in that application, in some embodiments, automatically determining one or more undercut regions of the virtual preparation tooth can occur during the automatic determining an insertion direction onto the virtual preparation tooth step 418 shown in FIG. 4.

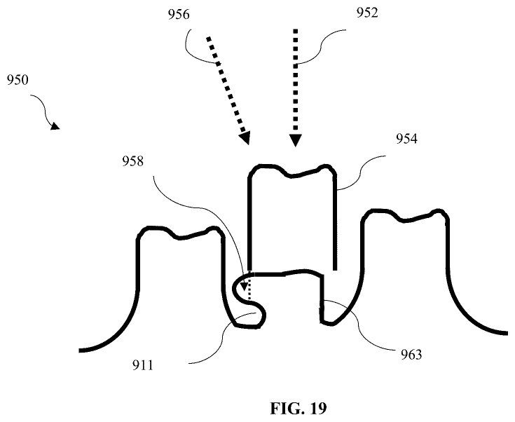

As illustrated in FIG. 19, in some embodiments, the computer implemented method can set a default path of insertion that is an optimal path of insertion to minimize undercuts 961. FIG. 19 illustrates a cross section illustration 950 of a portion of a patient's dentition in 2D. It is understood the patient's dentition is in 3D. In some embodiments, undercuts can represent one or more side wall regions that extend from the virtual tooth surface. An undercut 961 can cause one or more virtual preparation tooth side surface regions 958 of the virtual preparation tooth 963 to block the margin when viewed from the path of insertion 952 or 956, for example. This can cause restoration seating issues since the restoration 954 in some embodiments is to be arranged to maximally connect to the margin, for example. If any portion of the virtual margin is blocked along the path of insertion by one or more virtual preparation tooth side surface regions, then the computer-implemented method can determine that a virtual open margin exists. In some embodiments, an open margin can include one or more undercut regions blocking the margin along an insertion direction.

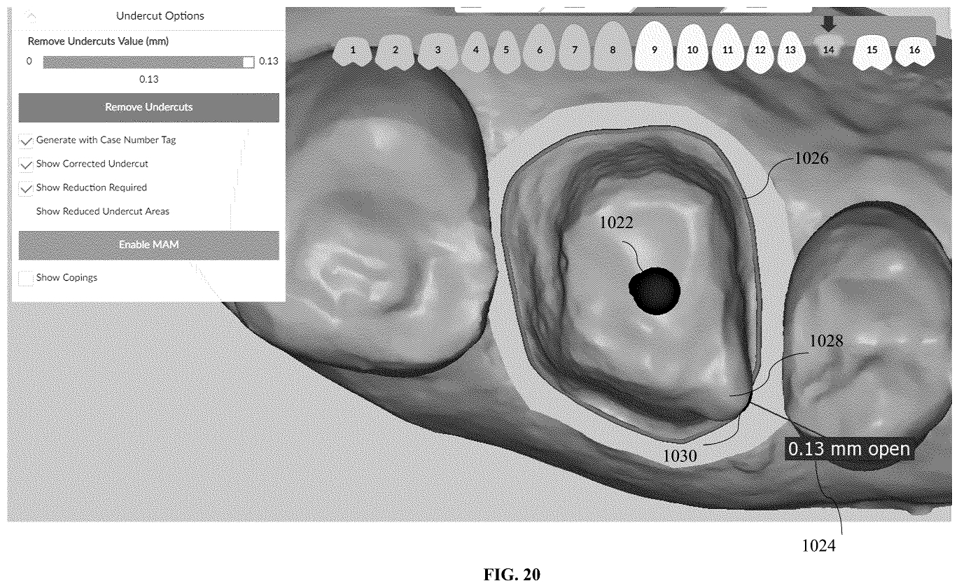

FIG. 20 illustrates an example of a virtual open margin. FIG. 20 illustrates a path of insertion 1022 having a virtual margin 1026. Due to the virtual preparation tooth side surface region 1039, the virtual preparation tooth has an open virtual margin 1028. The computer-implemented method can in some embodiments automatically determine a virtual preparation sidewall reduction value 1024 (also called an undercut value). The computer-implemented method can determine this value, for example, by determining how much of the sidewall is causing the open virtual margin 1028 when viewed along the path of insertion 1022. As illustrated in FIG. 20, in some embodiments, the computer-implemented method determines one or more virtual preparation tooth reduction regions as virtual preparation tooth side surface regions 1039 that block the virtual margin 1026 when viewed from the point of insertion 1022. The computer-implemented method can determine the virtual preparation tooth side surface reduction amount 1024 necessary to close the open virtual margin 1028 from the point of insertion 1022. In some embodiments, the computer-implemented method can determine one or more virtual preparation tooth side surface regions to reduce to close the margin. In some embodiments, the virtual preparation tooth side surface regions to reduce are virtual preparation tooth reduction regions.

In some embodiments one or more undercut regions can indicate a low quality physical preparation tooth. In some embodiments, one or more undercut regions preventing an insertion path can indicate a low quality preparation tooth, therefore requiring alteration of the physical preparation tooth. In some embodiments, one or more undercut regions creating an open margin can indicate a low quality physical preparation tooth, therefore requiring alteration of the physical preparation tooth.

In some embodiments the one or more physical preparation tooth issues can include automatically determining a clearance issue of a virtual restoration with a virtual opposing tooth. One or more examples of automatically determining automatically determining a clearance of a virtual restoration with a virtual opposing tooth can be found in U.S. application Ser. No. 16/918,586, the entirety of which was previously incorporated by reference. In some embodiments, determining the clearance issue can occur in the cement space step 420 of the automated design illustrated in FIG. 4. When a restoration is mounted to the physical preparation tooth, a minimum clearance is required to provide proper contact with an opposing tooth on the other jaw. In some embodiments a minimum clearance can include a cement space plus a minimum restoration thickness value.

In some embodiments, a lack of clearance with the virtual opposing tooth can mean a lower quality of the physical preparation tooth, therefore requiring alteration of the physical preparation tooth.

In some embodiments the one or more physical preparation tooth issues can include determining an margin line cannot be generated automatically. In some embodiments, determining the margin line cannot be generated automatically can occur in the margin AI step 416 in the automated design. In some embodiments determining the margin line cannot be generated automatically can be from an unclear scan or unclear physical margin line for the physical preparation tooth. In some embodiments determining the margin line cannot be generated automatically can be from an oversmooth physical margin. In some embodiments determining the margin line cannot be generated automatically can be from lack of curvature of the physical preparation tooth or the physical margin. In some embodiments, a probability of successfully generating the physical restoration is related to the an amount of the margin line that is determinable. The more of the margin line is determinable, the greater the probability of successfully generating the physical restoration. In some embodiments, the probability of successfully generating the physical restoration is high where it exceeds a user-configurable threshold. In some embodiments, margin line issues can also arise where the margin extends into neighboring teeth.

In some embodiments determining the margin line cannot be generated automatically can include a poor quality physical preparation tooth, therefore requiring alteration of the physical margin and/or the physical preparation tooth to increase the visibility of the physical margin and/or correct the margin line that extends into neighboring teeth.

Some embodiments of the computer-implemented method of virtual dental restoration design automation can include displaying virtually to the dentist or other user one or more physical preparation tooth issues detected while performing the automated virtual restoration design. In some embodiments, the virtual dental restoration design automation displaying virtually to the dentist or other user one or more physical preparation tooth issues detected while performing the automated virtual restoration design can be performed while the patient is in the dental office, for example during a patient visit and/or while the patient is receiving dental treatment in the dental chair. Displaying virtually to the dentist or other user one or more physical preparation tooth issues can provide the dentist and/or user with guidance and/or feedback on the quality of the physical preparation tooth. In some embodiments displaying virtually to the dentist or other user one or more physical preparation tooth issues can be performed in near-real time. In some embodiments displaying virtually to the dentist one or more physical preparation tooth issues can include highlighting one or more regions on the 3D virtual dental model needing reduction.

In some embodiments the computer-implemented method can display the issues on a computer display with a Graphical User Interface (“GUI”). In some embodiments displaying virtually to the dentist on a display one or more physical preparation tooth issues can include providing the dentist guidance on a virtual 3D dental model to correct one or more regions of the physical preparation tooth. This can be performed while the patient is still in the dental office and in the dental chair, in some embodiments. In some embodiments the guidance provides the dentist with insight regarding issues caused by their preparation of the physical preparation tooth. In some embodiments the guidance allows dentist to learn to reduce future occurrences of issues caused by their preparation of the physical preparation tooth.

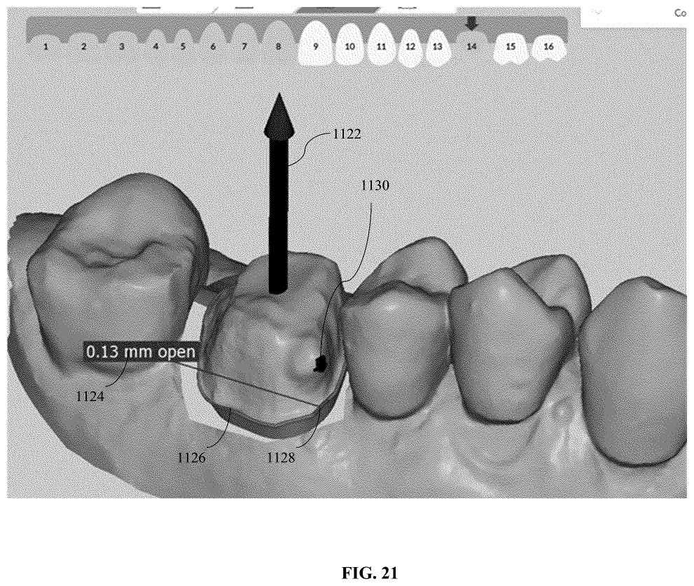

In some embodiments displaying virtually to the dentist one or more physical preparation tooth issues can includes displaying on a display one or more marked sidewall regions causing undercut regions on the virtual preparation tooth in the virtual dental model. As illustrated in FIG. 21, in some embodiments, the computer-implemented method can determine one or more virtual preparation tooth reduction regions as virtual preparation tooth side surface regions 1130 that block the virtual margin 1126 when viewed from the point of insertion 1122. The virtual preparation tooth side surface regions 1130 can indicated issues with insertion and proper seating of a physical restoration on the physical preparation tooth. The computer-implemented method can determine the virtual preparation tooth side surface reduction amount 1124 necessary to close the open virtual margin 1128 from the point of insertion 1122. In some embodiments, the computer-implemented method can determine and display one or more virtual preparation tooth side surface regions to reduce to close the margin along with the amount of reduction needed to close the margin. In some embodiments, the computer-implemented method can display to the dentist or other user one or more physical preparation tooth sidewall regions to reduce, as well as the amount to reduce. The virtual preparation tooth side surface regions to reduce correspond to physical preparation tooth reduction regions. Displaying the virtual preparation tooth side surface regions to a dentist or other user while the patient is receiving treatment can allow a dentist to make the reductions to the physical preparation tooth while the patient is still in the chair and in the same visit. This can decrease turnaround time by allowing the dentist to address physical preparation tooth issues in one visit so that the patient doesn't have to return to the office for further physical preparation tooth reductions to address issues with the physical preparation tooth.

In some embodiments displaying virtually to the dentist one or more physical preparation tooth issues can include displaying on a display one or more marked clearance issues on the virtual preparation tooth. In some embodiments, the one or more marked clearance issues can be detected during the cement space step of automated design. In some embodiments, the clearance issue can be related to the thickness of the restoration and the clearance between the physical preparation tooth and the physical opposing tooth.

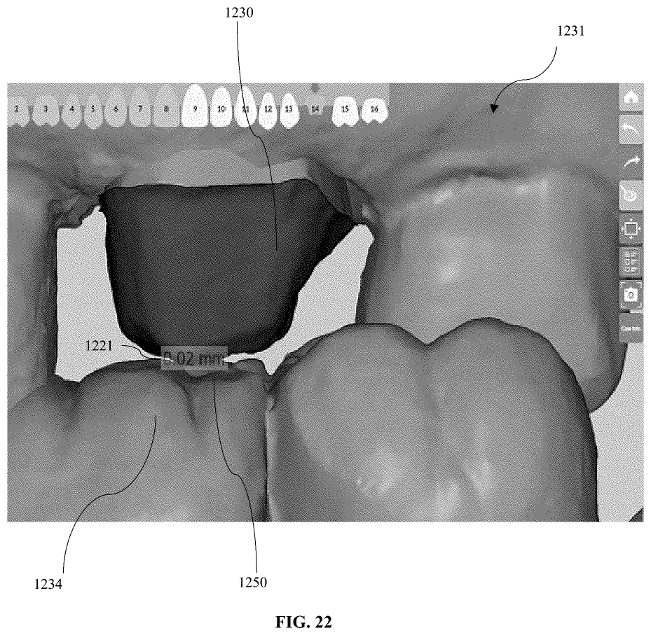

As illustrated in FIG. 22, in some embodiments, the computer-implemented method can detect an insufficient virtual occlusal clearance 1250 by determining a virtual occlusal clearance 1221 between one or more virtual preparation tooth occlusal surfaces and one or more virtual opposing tooth occlusal surfaces is less than the minimum required occlusal clearance. In some embodiments, the computer-implemented method determines the virtual occlusal clearance when a virtual jaw is closed or clenched, or when the virtual preparation tooth 1230 and virtual opposing tooth 1234 are closest to each other. The computer-implemented method in this way can determine that a height of the virtual preparation tooth 1230 and a height of the virtual opposing tooth 1234 are too large to accommodate the restoration's minimum restoration thickness between them when the jaw is closed or clenched.

To accommodate the restoration between the virtual preparation tooth 1230 and the virtual opposing tooth 1234, the computer-implemented method can determine a total virtual reduction amount necessary to satisfy the minimum required occlusal clearance. The computer-implemented method can display the total virtual reduction amount on a GUI to illustrate the total reduction necessary. In some embodiments, the total virtual reduction amount is a difference between the virtual occlusal clearance and the minimum required occlusal clearance, for example. In some embodiments, the computer-implemented method can determine a default virtual reduction value. For example, in some embodiments, the insufficient clearance can be an insufficient occlusal clearance 1250 between an occlusal surface of the virtual preparation tooth 1230 and an occlusal surface of a virtual opposing tooth 1234.

In some embodiments the physical preparation tooth can be modified by the dentist to address the one or more physical preparation tooth issues. Some embodiments can include allowing a dentist to modify the physical preparation tooth (and/or opposing or surrounding teeth/dentition), rescan at least a portion of the patient's dentition including the modified physical preparation tooth and/or opposing tooth and/or surrounding dentition to generate a modified 3D virtual dental model including a modified virtual preparation tooth and/or opposing tooth and/or surrounding dentition, and uploading the modified 3D virtual dental model. In some embodiments the modified 3D virtual dental model is used for the same case, without generating a new case.

Once the dentist has made any necessary modifications to the physical preparation tooth/opposing tooth/surrounding dentition, the computer implemented method can further include receiving a modified 3D virtual dental model including at least one modified virtual preparation tooth, and/or at least one modified virtual opposing and/or modified dentition, the modified virtual preparation tooth including a virtual representation of a modified physical preparation tooth modified by a dentist in some embodiments. In some embodiments, the modifications by the dentist, rescans, and re-uploading are performed while the patient is in the dental chair receiving treatment.

Some embodiments of the computer-implemented method of virtual dental restoration design automation can include displaying virtually to the dentist or other user a generated virtual restoration for one or more adjustments where no physical preparation tooth issues are detected while performing the automated virtual restoration design. In some embodiments, displaying virtually to the dentist or other user a generated virtual restoration for one or more adjustments where no physical preparation tooth issues are detected while performing the automated virtual restoration design can be performed while the patient is in the dental office.

In some embodiments, the one or more adjustments can include adjusting the virtual margin line. In some embodiments, the one or more adjustments can include adjusting a virtual contact with an opposing tooth. In some embodiments, the one or more adjustments can include adjusting a virtual contact with one or more teeth adjacent to the preparation tooth. In some embodiments, the adjustments can include adjustments to the margin line or adjustments to the virtual restoration. The adjustments can also be referred to as “design check”, or “DC”.

Some embodiments can include performing adjustments, or DC on the generated 3D virtual dental restoration model. In some embodiments, the computer-implemented method can display at least a portion of the 3D virtual model of the patient's dentition that includes the generated 3D virtual dental restoration model on a display such as a computer screen in a Graphical User Interface (“GUI”) that can include interactive controls that can allow a dental technician, dentist, or other user to manipulate one or more features of the generated 3D virtual dental restoration model.

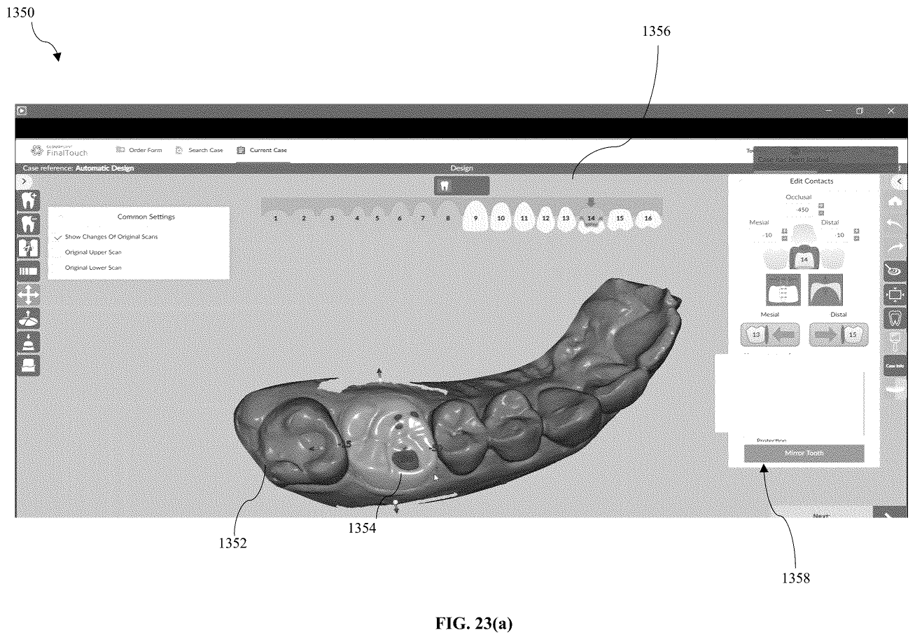

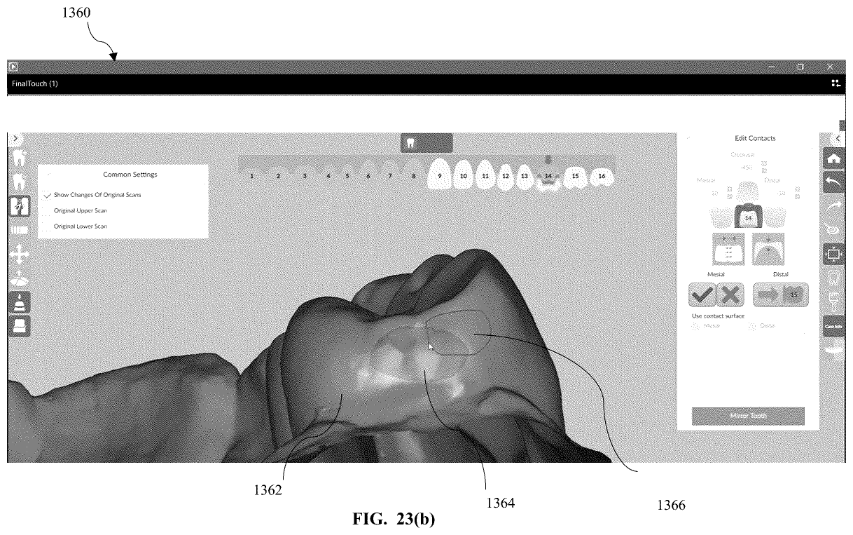

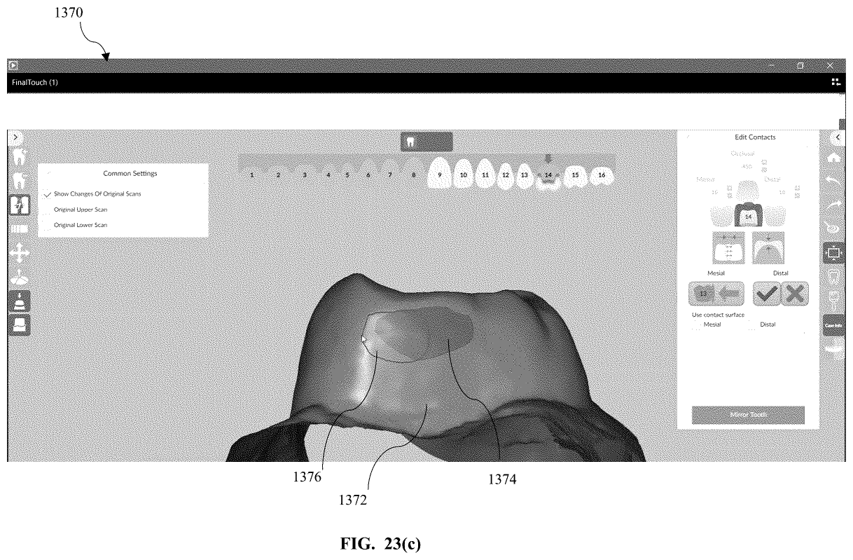

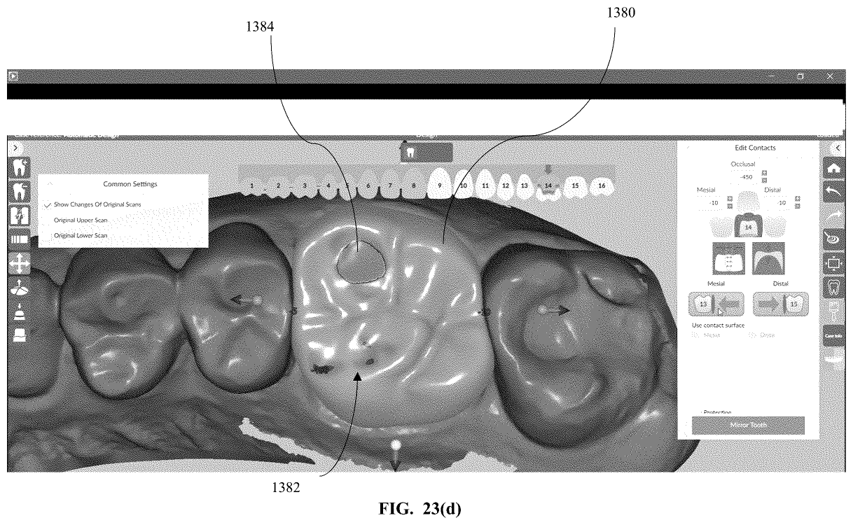

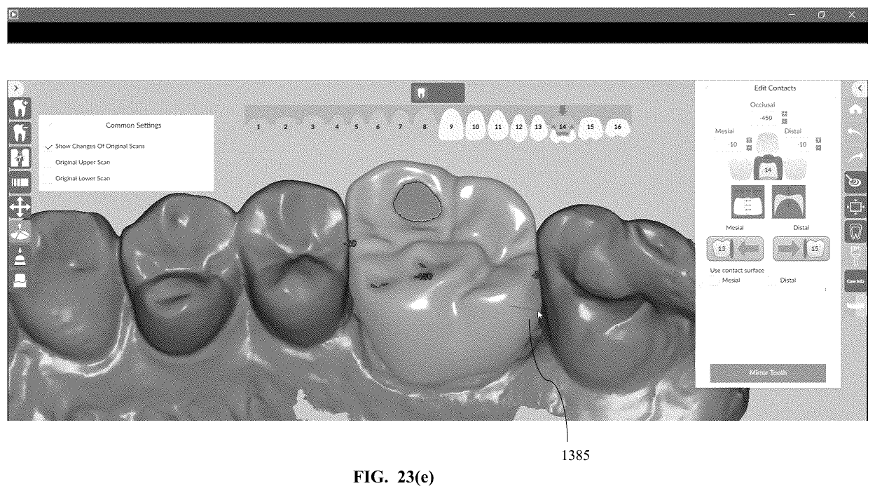

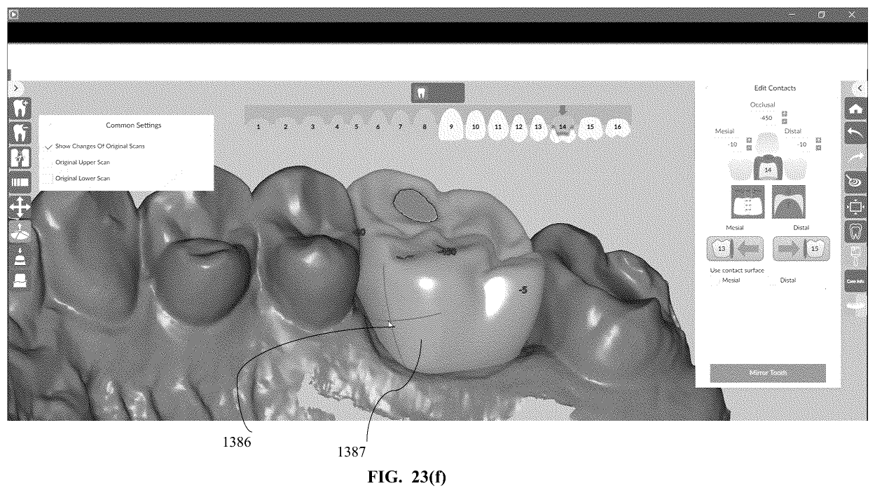

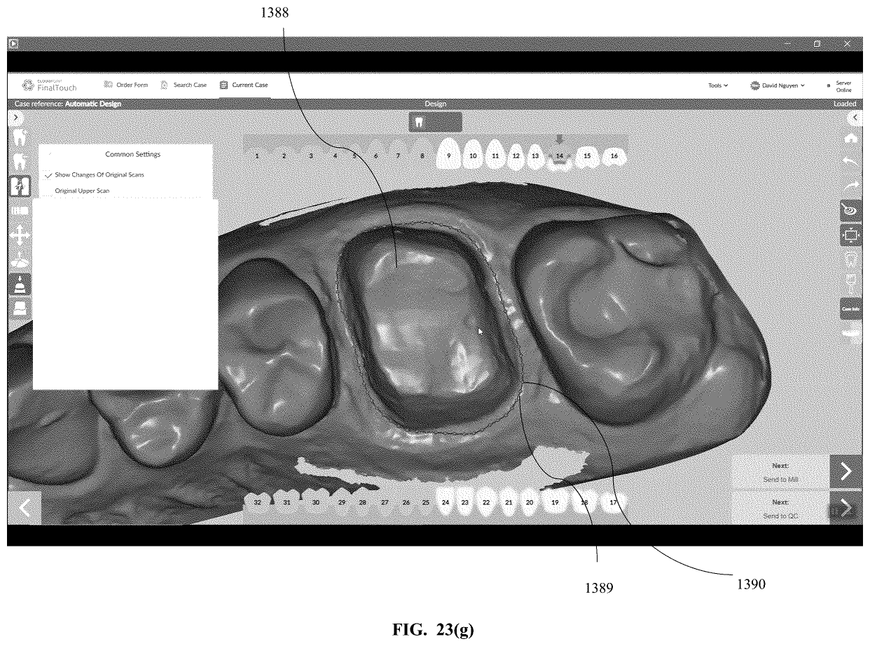

FIG. 23(a) illustrates an example of a GUI 1350 that can be used as part of the DC process in some embodiments. In the example, a generated 3D virtual restoration model is loaded for DC. The DC process can display at least a portion of the 3D virtual dental model 1352 of a patient's dentition as well as a generated 3D virtual dental restoration model 1354 in the GUI 1350 along with information regarding the 3D virtual dental restoration model 1354 and its location and orientation with respect to surrounding dentition. For example, in some embodiments, the computer-implemented method can display and indicate a virtual tooth number 1356 along with its neighboring virtual teeth in a representation of the upper or lower jaw. In some embodiments, the DC process can display information regarding the virtual tooth 1354 and its neighboring virtual teeth in a panel such as panel 1358, or other suitable GUI display element known in the art. The panel 1358 can provide information regarding the occlusal, mesial, and distal relationships of the generated 3D virtual dental restoration model with respect to surrounding dentition, for example. The GUI 1350 can provide one or control features to adjust the 3D virtual dental restoration model 1354 in some embodiments. For example, in some embodiments, the DC process can provide controls to adjust contact points the automatically generated 3D virtual dental restoration has with neighboring virtual teeth in the 3D virtual dental model.

In some embodiments, the DC process can provide GUI controls to adjust contact points such as mesial, distal, and/or occlusal contact points, for example. Mesial and distal contact points can be between the generated 3D virtual dental restoration model and neighboring virtual teeth in the 3D virtual dental model. Occlusal contact points can between an occlusal surface of the generated 3D virtual dental restoration model and an opposing virtual tooth on an opposing virtual jaw. FIG. 23(b) illustrates an example of adjusting mesial contact points in some embodiments. As illustrated in the figure, GUI 1360 can display at least a portion of the 3D virtual dental model along with a mesial side of a generated 3D virtual dental restoration model 1362. The GUI 1360 can display a mesial contact surface region 1364 between the 3D virtual dental restoration model 1362 and its mesial neighboring tooth (not shown) in the 3D virtual dental model. The GUI 1360 can provide an adjustment tool that can allow a user to select using an input device a mesial adjustment surface region 1366 that the computer-implemented method can reduce, for example. In some embodiments, the adjustment can decrease the size of the adjustment surface region such as adjustment surface region 1366. In some embodiments, the adjustment can increase the size of the adjustment surface region 1366. FIG. 23(c) illustrates an example of adjusting distal contact points in some embodiments. As illustrated in the figure, GUI 1370 can display at least a portion of the 3D virtual dental model along with a distal side of a generated 3D virtual dental restoration model 1372. The GUI 1370 can display a distal contact surface region 1374 between the 3D virtual dental restoration model 1372 and its distal neighboring tooth (not shown) in the 3D virtual dental model. The GUI 1370 can provide an adjustment tool that can allow a user to select using an input device a distal adjustment surface region 1376 that the computer-implemented method can adjust, for example. In some embodiments, the adjustment can decrease the size of the adjustment surface region such as adjustment surface region 1376. In some embodiments, the adjustment can increase the size of the adjustment surface region 1376.

In some embodiments, the DC process can provide GUI controls allowing a user to adjust the occlusal contact points. For example, FIG. 23(d) illustrates a generated 3D virtual dental restoration model 1380 as viewed from the occlusal direction, showing an occlusal surface such as occlusal surface 1382. In some embodiments, the computer-implemented method can provide one or more virtual tools to adjust an occlusion contact region, such as occlusal contact region 1384. In some embodiments, the adjustment can decrease the size of the adjustment surface region such as occlusal contact region 1384. In some embodiments, the adjustment can increase the size of the adjustment surface region 1384.

In some embodiments, the DC process can provide GUI controls allowing a user to adjust the shape or contour of the automatically generated 3D virtual dental restoration as illustrated in FIG. 23(e) and FIG. 23(f), for example. In the example of FIG. 23(e), a user can select a contour region such as contour region 1385 to adjust. In the example of FIG. 23(f), a user can select a contour region such as contour region 1387. In some embodiments, the contour control can include a visual indicator that can define the contour region. One example of such a visual indicator can include an “X” or hash mark whose size can be adjusted by a user. One example of such a visual indicator is visual indicator 1386. Upon hovering a pointer over the generated 3D virtual dental restoration, the visual indicator can appear on at least a portion of the generated 3D virtual dental restoration model in some embodiments. The user can adjust the shape or contour of the contour region in some embodiments by pressing a button on an input device such as a mouse and dragging the contour region in a desired direction, as an example. Other GUI controls known in the art can be used to allow a user to adjust the shape or contour of the automatically generated 3D virtual dental restoration in some embodiments.

In some embodiments, the DC process can display at least a portion of the 3D virtual dental model 1388 of a patient's dentition and the automatically determined margin line proposal and allow a user to modify the determined margin line. FIG. 23(g) illustrates an example in which the DC process displays the determined margin line proposal 1389 in a GUI. In some embodiments, the 3D virtual dental restoration is hidden or not displayed so that the determined margin line is visible. In some embodiments, the GUI can provide virtual handles 1390 to allow a user to adjust the automatically determined margin line. This can advantageously allow, for example, correction of the automatically determined margin line as part of the DC process.

In some embodiments, once changes to the generated 3D virtual dental restoration model as part of the DC process are complete, the computer-implemented method can apply the changes to the 3D virtual dental restoration model to provide a modified 3D virtual dental restoration model. In some embodiments, the changes are applied as they are made to provide visual feedback. In some embodiments, where the changes to the model are major or fundamental, the computer-implemented method can regenerate the 3D virtual dental restoration using the generating neural network. In some embodiments, major and/or fundamental changes can include, but are not limited to, for example, changes to the margin line proposal. In some embodiments, major and/or fundamental changes can be based on a user configurable value of change as measured geometrically in the model, for example. In some embodiments, the computer-implemented method can, for the regenerated 3D virtual dental restoration model, perform one or more features described herein, and provide the regenerated 3D virtual dental restoration model for DC processing. In some embodiments, where the DC changes are not major or fundamental, the computer-implemented method can apply the changes to the virtual margin line and the generated 3D virtual dental restoration model without regeneration. For example, in some embodiments, upon receiving minor adjustments to the margin line, the computer-implemented method can include adjusting the virtual margin line and/or the virtual restoration as needed. In some embodiments, upon receiving minor adjustments to the virtual restoration, the computer-implemented method can include applying the minor adjustments to the virtual restoration without regenerating the virtual restoration. In some embodiments, the computer-implemented method can include determining one or more physical preparation tooth issues with the regenerated virtual restoration. In some embodiments, any adjustments can be made by a dentist in a dental office while the patient is in the chair receiving treatment.

Some embodiments can include tracking a performance of the dentist based on the quality of one or more physical preparation teeth. Some embodiments can include tracking a performance of the dentist based on the quality of one or more physical preparation teeth. Some embodiments can include tracking a performance of a dental office comprising one or more dentists based on the quality of the one or more physical preparation teeth prepared in the dental office by the one or more dentists. In some embodiments, the tracking comprises analytics of dentist and dental office and performance over a given time period. In some embodiments, the analytics comprise one or more of the following: order ID, tooth ID, patient ID, patient name, doctor name, comments, mill/fabrication location, restoration type, material, origin of scan files (scanner, computer, or saved scans), and guidance information. In some embodiments, guidance information comprises number of scanning sessions taken (rescans), duration of scans, type of issue discovered for each scan, resolution taken for each scan (how solved). In some embodiments, scan data is recorded and not overridden during rescans. Some embodiments can include developing a baseline level of performance. In some embodiments, the baseline level of performance comprises number of physical preparations causing issues. Some embodiments can include curating educational content based on the type of issues typically encountered when preparing a preparation tooth.

In some embodiments, the analytics and guidance information captured can be provided as aggregated feedback to the dentist, clinician, and the practice. In some embodiments, the analytics, guidance, and other information can be provided to show a trend overtime. In some embodiments, providing this very short, closed feedback loop can advantageously improve the preparation tooth/teeth over time. The analytics, guidance, and information along with any aggregated feedback can provide progress reports, which can help DSOs work with dentists and doctors who need to improve to meet internal performance metrics.

Some embodiments can include providing individual dentists with feedback regarding their specific preparation techniques to minimize future preparation issues. Some embodiments can include incorporating the educational content into a continuing education program for dentists. In some embodiments, the performance comprises a scorecard. In some embodiments, the scorecard comprising guidance information is displayed after each treatment session. In some embodiments, a dashboard shows application usage and overall performance. In some embodiments, the scorecard can indicate success/failure rate over a period of time. In some embodiments, the scorecard can provide success/failure rate with respect to one or more other dentists. In some embodiments, the one or more other dentists are in the same dental office. In some embodiments, the dental office can include a DSO. Some embodiments can include providing a scorecard summary to the DSO. This can help the DSO identify areas in which dentists may need more training or guidance, and also be used to determine performance of individual dentists, for example.

Some embodiments can include milling a physical 3D virtual restoration from the successfully generated 3D virtual restoration. In some embodiments the manufacturing facility can be a dental laboratory. In some embodiments the manufacturing facility performs a manufacturing check. In some embodiments, the dental laboratory or other manufacturing facility is located external to the dental office.

In some embodiments milling can be a computer aided manufacturing process (“CAM”). Some embodiments can include staging one or physical restorations. In some embodiments staging can include grouping one or more physical restorations by patient for shipping. Some embodiments can include bundling one or more physical restorations. In some embodiments bundling can include grouping together one or more restorations for a dental office together for shipping to the dental office.

Some embodiments can include routing the virtual 3D virtual restoration model to a computer aided manufacturing (“CAM”) process. In some embodiments the CAM process can be performing a design machinability check. In some embodiments the design machinability check can include the following steps, not necessarily limited to this order: (1). generating a virtual milled restoration from NC-tile (milling strategy). In some embodiments the result of this stage can be a milling surface that should coincide with real milled crown. In some embodiments the virtually milled restoration does not comprise one or more undercuts. (2). comparing the virtually milled restoration with the virtual restoration design. In some embodiments comparing can be comparing outer and inner surfaces of the virtually milled restoration and occlusal table. In some embodiments if a difference exists between virtually milled restoration and the virtual restoration design, then the design machinability check fails. In some embodiments upon failure of the design machinability check, routing the virtual restoration to manual milling. In some embodiments if no difference exists between the virtually milled restoration and the virtual restoration design, then the auto milling check passes. In some embodiments upon success of the auto milling check, routing the virtual restoration to automatic milling. In some embodiments automatic milling comprises: (a) Nesting (b) Determining block based on enlargement factor and shade (c) Milling restoration (such as a crown, for example) (d) Determining Staining/shade—gingival and occlusal surface (e) Sintering the restoration.

In some embodiments failure of machinability can be unmillable undercuts, restoration size too big for blocks, or shade unavailability. In some embodiments upon failure of machinability check, routing the virtual restoration design for manual machining. In some embodiments upon success of machinability check, routing the virtual design restoration to automatic machining. In some embodiments upon passing the design machinability check, the CAM process routes the virtual 3D virtual restoration model to an automatic mill to perform automatic milling. In some embodiments upon completing automatic milling, performing virtual milling DC. In some embodiments upon passing virtual milling DC, performing staging.

One or more advantages of one or more features can include, for example, saving overall turnaround time, reducing chairside time, improved patient experience by avoiding the patient coming back for multiple treatments. One or more advantages of one or more features can include, for example, reducing or eliminating scrapping of physical restorations due to one or more issues such as clearance, insertion, and/or undercuts. One or more advantages of one or more features can include, for example, improved quality of physical restorations. One or more advantages of one or more features can include, for example, fewer remakes of physical restorations. One or more advantages of one or more features can include, for example, fewer trips by patients to the dentist or other user's office and reducing the number of times the patient is put under anesthesia. One or more advantages of one or more features can include, for example, providing a dentist with near real-time feedback regarding the physical preparation tooth to allow corrections while treating a patient chairside. One or more advantages of one or more features can include, for example, allowing a dentist to prepare a tooth and generate a virtual restoration for that tooth in a single office visit. One or more advantages of one or more features can include, for example, improved (faster) turnaround time for getting physical restoration. One or more advantages of one or more features can include, for example, reduced chair time for the patient. One or more advantages can include, for example, reduction in scrapped restorations due to one or more issues optionally including but not limited to undercut issues, clearance issues, and/or open margin issues.

Some embodiments include a processing system for virtual dental restoration design automation that can include: a processor, a computer-readable storage medium including instructions executable by the processor to perform steps including: receiving a 3D virtual dental model of at least a portion of a patient's dentition, the 3D virtual dental model comprising at least one virtual preparation tooth, the virtual preparation tooth comprising a digital representation of a physical preparation tooth prepared by a dentist; performing an automated virtual restoration design using the 3D virtual dental model; displaying virtually to the dentist one or more physical preparation tooth issues detected while performing the automated virtual restoration design; and displaying virtually to the dentist a generated virtual restoration for one or more adjustments where no physical preparation tooth issues are detected while performing the automated virtual restoration design.

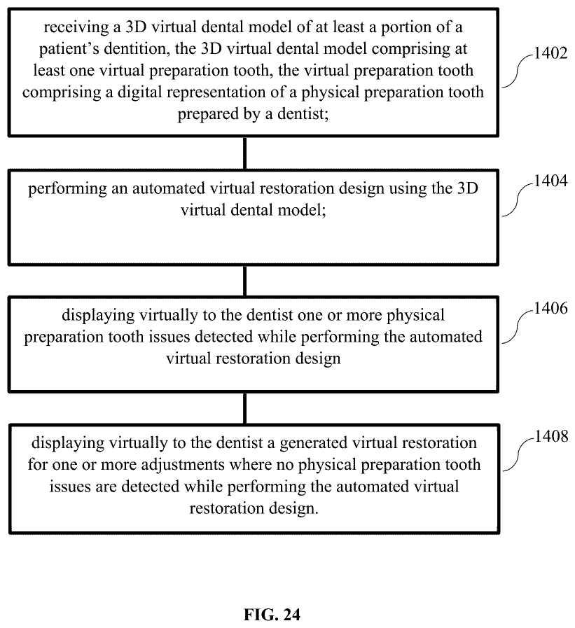

FIG. 24 is a flowchart illustrating one or more steps in some embodiments. The steps can include receiving a 3D virtual dental model of at least a portion of a patient's dentition, the 3D virtual dental model comprising at least one virtual preparation tooth, the virtual preparation tooth comprising a digital representation of a physical preparation tooth prepared by a dentist at 1402; performing an automated virtual restoration design using the 3D virtual dental model at 1404; displaying virtually to the dentist one or more physical preparation tooth issues detected while performing the automated virtual restoration design at 1406; and displaying virtually to the dentist a generated virtual restoration for one or more adjustments where no physical preparation tooth issues are detected while performing the automated virtual restoration design at 1408.

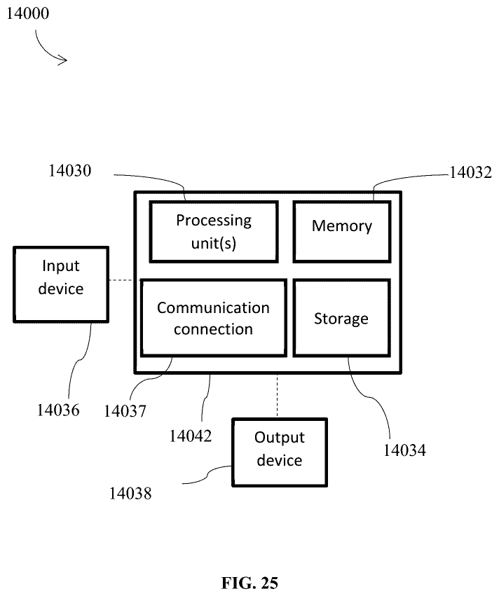

FIG. 25 illustrates a processing system 14000 in some embodiments. The system 14000 can include a processor 14030, computer-readable storage medium 14034 having instructions executable by the processor to perform one or more steps described in the present disclosure. In some embodiments, one or more features can be performed by a user using an input device while viewing the virtual model on a display, for example. In some embodiments, the computer-implemented method can allow the input device to manipulate the virtual model displayed on the display. For example, in some embodiments, the computer-implemented method can rotate, zoom, move, and/or otherwise manipulate the virtual model in any way as is known in the art.

In some embodiments, one or more virtual surfaces of a 3D virtual model can be selected on a virtual tooth or other region using an input device whose pointer is shown on a display, for example. The pointer can be used to select a region of one point by clicking on an input device such as a mouse or tapping on a touch screen for example. A virtual surface of multiple points can be selected by dragging the pointer across a virtual surface in some embodiments, for example. Other techniques known in the art can be used to select a point or virtual surface.

In some embodiments the computer-implemented method can display a virtual model on a display and receive input from an input device such as a mouse or touch screen on the display for example. The computer-implemented method can, upon receiving manipulation commands, rotate, zoom, move, and/or otherwise manipulate the virtual model in any way as is known in the art in some embodiments.

One or more of the features disclosed herein can be performed and/or attained automatically, without manual or user intervention. One or more of the features disclosed herein can be performed by a computer-implemented method. The features-including but not limited to any methods and systems-disclosed may be implemented in computing systems. For example, the computing environment 14042 used to perform these functions can be any of a variety of computing devices (e.g., desktop computer, laptop computer, server computer, tablet computer, gaming system, mobile device, programmable automation controller, video card, etc.) that can be incorporated into a computing system comprising one or more computing devices. In some embodiments, the computing system may be a cloud-based computing system.

For example, a computing environment 14042 may include one or more processing units 14030 and memory 14032. The processing units execute computer-executable instructions. A processing unit 14030 can be a central processing unit (CPU), a processor in an application-specific integrated circuit (ASIC), or any other type of processor. In some embodiments, the one or more processing units 14030 can execute multiple computer-executable instructions in parallel, for example. In a multi-processing system, multiple processing units execute computer-executable instructions to increase processing power. For example, a representative computing environment may include a central processing unit as well as a graphics processing unit or co-processing unit. The tangible memory 14032 may be volatile memory (e.g., registers, cache, RAM), non-volatile memory (e.g., ROM, EEPROM, flash memory, etc.), or some combination of the two, accessible by the processing unit(s). The memory stores software implementing one or more innovations described herein, in the form of computer-executable instructions suitable for execution by the processing unit(s).

A computing system may have additional features. For example, in some embodiments, the computing environment includes storage 14034, one or more input devices 14036, one or more output devices 14038, and one or more communication connections 14037. An interconnection mechanism such as a bus, controller, or network, interconnects the components of the computing environment. Typically, operating system software provides an operating environment for other software executing in the computing environment, and coordinates activities of the components of the computing environment.

The tangible storage 14034 may be removable or non-removable, and includes magnetic or optical media such as magnetic disks, magnetic tapes or cassettes, CD-ROMs, DVDs, or any other medium that can be used to store information in a non-transitory way and can be accessed within the computing environment. The storage 14034 stores instructions for the software implementing one or more innovations described herein.

The input device(s) may be, for example: a touch input device, such as a keyboard, mouse, pen, or trackball; a voice input device; a scanning device; any of various sensors; another device that provides input to the computing environment; or combinations thereof. For video encoding, the input device(s) may be a camera, video card, TV tuner card, or similar device that accepts video input in analog or digital form, or a CD-ROM or CD-RW that reads video samples into the computing environment. The output device(s) may be a display, printer, speaker, CD-writer, or another device that provides output from the computing environment.

The communication connection(s) enable communication over a communication medium to another computing entity. The communication medium conveys information, such as computer-executable instructions, audio or video input or output, or other data in a modulated data signal. A modulated data signal is a signal that has one or more of its characteristics set or changed in such a manner as to encode information in the signal. By way of example, and not limitation, communication media can use an electrical, optical, RF, or other carrier.

Any of the disclosed methods can be implemented as computer-executable instructions stored on one or more computer-readable storage media 14034 (e.g., one or more optical media discs, volatile memory components (such as DRAM or SRAM), or nonvolatile memory components (such as flash memory or hard drives)) and executed on a computer (e.g., any commercially available computer, including smart phones, other mobile devices that include computing hardware, or programmable automation controllers) (e.g., the computer-executable instructions cause one or more processors of a computer system to perform the method). The term computer-readable storage media does not include communication connections, such as signals and carrier waves. Any of the computer-executable instructions for implementing the disclosed techniques as well as any data created and used during implementation of the disclosed embodiments can be stored on one or more computer-readable storage media 14034. The computer-executable instructions can be part of, for example, a dedicated software application or a software application that is accessed or downloaded via a web browser or other software application (such as a remote computing application). Such software can be executed, for example, on a single local computer (e.g., any suitable commercially available computer) or in a network environment (e.g., via the Internet, a wide-area network, a local-area network, a client-server network (such as a cloud computing network), or other such network) using one or more network computers.

For clarity, only certain selected aspects of the software-based implementations are described. Other details that are well known in the art are omitted. For example, it should be understood that the disclosed technology is not limited to any specific computer language or program. For instance, the disclosed technology can be implemented by software written in C++, Java, Perl, Python, JavaScript, Adobe Flash, or any other suitable programming language. Likewise, the disclosed technology is not limited to any particular computer or type of hardware. Certain details of suitable computers and hardware are well known and need not be set forth in detail in this disclosure.

It should also be well understood that any functionality described herein can be performed, at least in part, by one or more hardware logic components, instead of software. For example, and without limitation, illustrative types of hardware logic components that can be used include Field-programmable Gate Arrays (FPGAs), Program-specific Integrated Circuits (ASICs), Program-specific Standard Products (ASSPs), System-on-a-chip systems (SOCs), Complex Programmable Logic Devices (CPLDs), etc.

Furthermore, any of the software-based embodiments (comprising, for example, computer-executable instructions for causing a computer to perform any of the disclosed methods) can be uploaded, downloaded, or remotely accessed through a suitable communication means. Such suitable communication means include, for example, the Internet, the World Wide Web, an intranet, software applications, cable (including fiber optic cable), magnetic communications, electromagnetic communications (including RF, microwave, and infrared communications), electronic communications, or other such communication means.

In view of the many possible embodiments to which the principles of the disclosure may be applied, it should be recognized that the illustrated embodiments are only examples and should not be taken as limiting the scope of the disclosure.

What is claimed is:

1. A computer-implemented method of virtual dental restoration design automation, comprising:
receiving a 3D virtual dental model of at least a portion of a patient's dentition, the 3D virtual dental model comprising at least one virtual preparation tooth, the virtual preparation tooth comprising a digital representation of a physical preparation tooth prepared by a dentist;
performing an automated virtual restoration design using the 3D virtual dental model;
displaying virtually to the dentist one or more physical preparation tooth issues detected while performing the automated virtual restoration design; and
displaying virtually to the dentist a generated virtual restoration for one or more adjustments where no physical preparation tooth issues are detected while performing the automated virtual restoration design.

2. The method of claim 1, wherein the automated virtual restoration design comprises one or more selected from the group consisting of decimation of the 3D virtual dental model, meshing, segmentation, determining an occlusal direction, determining a bite alignment, preparation die localization, determining a buccal direction, determining a margin for the at least one virtual preparation tooth, determining an insertion direction onto the virtual preparation tooth, determining cement space for the virtual preparation tooth, generating the virtual restoration, and pulling the virtual restoration to the margin.

3. The method of claim 1, wherein the one or more physical preparation tooth issues comprises one or more undercut regions.

4. The method of claim 1, wherein the one or more physical preparation tooth issues comprises a lack of clearance.

5. The method of claim 1, wherein the one or more physical preparation tooth issues comprises a lack of insertion direction.

6. The method of claim 1, wherein the one or more physical preparation tooth issues comprises a margin line cannot be generated automatically.

7. The method of claim 1, wherein displaying the one or more physical preparation tooth issues comprises highlighting one or more regions on the 3D virtual dental model needing reduction.

8. The method of claim 1, further comprising illustrating an insertion direction to the dentist upon an automated design success.

9. The method of claim 1, wherein one or more steps are performed during a single patient visit.

10. The method of claim 9, wherein the patient is in the dental chair receiving dental treatment.

11. The method of claim 1, further comprising upon detecting one or more issues during automated design, allowing a dentist to modify the physical preparation tooth, rescan at least a portion of the patient's dentition comprising the modified physical preparation tooth to generate a modified 3D virtual dental model comprising a modified virtual preparation tooth, and uploading the modified 3D virtual dental model.

12. The method of claim 1, wherein the generated virtual restoration is adjustable by the dentist.

13. The method of claim 1, further comprising determining a quality of the one or more physical preparation teeth based on the automated design process.

14. The method of claim 13, wherein the automated design error indicates a lower quality physical preparation tooth.

15. The method of claim 1, further comprising tracking a performance of the dentist based on the quality of one or more physical preparation teeth.

16. The method of claim 1, further comprising routing the 3D virtual restoration to a computer aided manufacturing (“CAM”) process, wherein the CAM process comprises performing a design machinability check for automated milling.

17. The method of claim 16, further comprising milling the virtual restoration.

18. A non-transitory computer readable medium storing executable computer program instructions to provide virtual dental restoration design automation, the computer program instructions comprising instructions for:
receiving a 3D virtual dental model of at least a portion of a patient's dentition, the 3D virtual dental model comprising at least one virtual preparation tooth, the virtual preparation tooth comprising a digital representation of a physical preparation tooth prepared by a dentist;
performing an automated virtual restoration design using the 3D virtual dental model;
displaying virtually to the dentist one or more physical preparation tooth issues detected while performing the automated virtual restoration design; and
displaying virtually to the dentist a generated virtual restoration for one or more adjustments where no physical preparation tooth issues are detected while performing the automated virtual restoration design.

19. A system for virtual dental restoration design automation, the system comprising:
a processor; and
a non-transitory computer-readable storage medium comprising instructions executable by the processor to perform steps comprising:
receiving a 3D virtual dental model of at least a portion of a patient's dentition, the 3D virtual dental model comprising at least one virtual preparation tooth, the virtual preparation tooth comprising a digital representation of a physical preparation tooth prepared by a dentist;
performing an automated virtual restoration design using the 3D virtual dental model;
displaying virtually to the dentist one or more physical preparation tooth issues detected while performing the automated virtual restoration design; and
displaying virtually to the dentist a generated virtual restoration for one or more adjustments where no physical preparation tooth issues are detected while performing the automated virtual restoration design.
